SEQUENCE LISTING (22 sequences)

SEQ ID NO: 1 - protein, 12 aa, Artificial Sequence
  synthesized
KAFDITYVRLKF

SEQ ID NO: 2 - protein, 4 aa, Artificial Sequence
  synthesized
SDKP

SEQ ID NO: 3 - DNA, 20 bp, Artificial Sequence
  synthesized
gctgaaccaaggagacggaa

SEQ ID NO: 4 - DNA, 20 bp, Artificial Sequence
  synthesized
agaagttggcatggtagccc

SEQ ID NO: 5 - DNA, 20 bp, Artificial Sequence
  synthesized
atgtagttgccacgcacaga

SEQ ID NO: 6 - DNA, 20 bp, Artificial Sequence
  synthesized
ggggacagcgacacctttta

SEQ ID NO: 7 - DNA, 22 bp, Artificial Sequence
  synthesized
agacacaccagagataccatga

SEQ ID NO: 8 - DNA, 20 bp, Artificial Sequence
  synthesized
gaggacctgatccgtccaca

SEQ ID NO: 9 - DNA, 20 bp, Artificial Sequence
  synthesized
tctgaagagtgggcgtagga

SEQ ID NO: 10 - DNA, 20 bp, Artificial Sequence
  synthesized
ggctatttggggccatcgta

SEQ ID NO: 11 - DNA, 20 bp, Artificial Sequence
  synthesized
ttgagtccggcagacaatcc

SEQ ID NO: 12 - DNA, 20 bp, Artificial Sequence
  synthesized
ccttatccacgcgaatgacg

SEQ ID NO: 13 - DNA, 20 bp, Artificial Sequence
  synthesized
gagttggcagtgcaatacct

SEQ ID NO: 14 - DNA, 20 bp, Artificial Sequence
  synthesized
gccgtccttctcaaagttgt

SEQ ID NO: 15 - DNA, 20 bp, Artificial Sequence
  synthesized
acggcatggatctcaaagac

SEQ ID NO: 16 - DNA, 20 bp, Artificial Sequence
  synthesized
agatagcaaatcggctgacg

SEQ ID NO: 17 - DNA, 20 bp, Artificial Sequence
  synthesized
atgcggatcaaacctcacca

SEQ ID NO: 18 - DNA, 20 bp, Artificial Sequence
  synthesized
ccgctctgaacaaggctcac

SEQ ID NO: 19 - DNA, 19 bp, Artificial Sequence
  synthesized
tgaagcaggcatctgaggg

SEQ ID NO: 20 - DNA, 20 bp, Artificial Sequence
  synthesized
cgaaggtggaagagtgggag

SEQ ID NO: 21 - DNA, 20 bp, Artificial Sequence
  synthesized
ctcgctggacgttggaggaa

SEQ ID NO: 22 - DNA, 20 bp, Artificial Sequence
  synthesized
cacgcgcaagaaccatcact

(19) United States
(12) Patent Application Publication
(10) Pub. No.: US 2016/0024249 A1
(43) Pub. Date: Jan. 28, 2016

(54) Temperature-Responsive Polymer Compositions and Methods of Use

(71) Applicant: Vanderbilt University, Nashville, TN (US)

(72) Inventors: Hak-Joon Sung, Nashville, TN (US); Xintong Wang, Nashville, TN (US); Mukesh K. Gupta, Nashville, TN (US); Timothy C. Boire, Nashville, TN (US)

(21) Appl. No.: 14/806,642

(22) Filed: Jul. 22, 2015

Related U.S. Application Data
(60) Provisional application No. 62/027,706, filed on Jul. 22, 2014.

(51) Int. Cl. C08G 63/91; A61K 38/10; A61K 47/48; A61K 9/00; A61K 38/07

(52) U.S. Cl. CPC C08G 63/91; A61K 9/0024; A61K 38/07; A61K 47/48215; A61K 38/10

(57) ABSTRACT

Embodiments of the invention include polymeric compounds that comprise a co-polymer microgel at least one bioactive agent, pharmaceutical compounds comprising compounds of the present invention, and methods for treating damaged tissue to a patient in need thereof by administering by injection an effect amount of the composition.

### PRIOR APPLICATIONS

This application claims benefit to Patent Application No. 62/027,706, the contents of which are incorporated herein by reference.

### GOVERNMENT SUPPORT

This invention was made with government support under NIH HL091465, NSF DMR 1006558, and HL104040 awarded by The National Institutes of Health. The government has certain rights in the invention.

### TECHNICAL FIELD

The presently-disclosed subject matter relates to temperature-responsive polymer compositions. In particular, the presently-disclosed subject matter relates to compositions that can deliver cells, therapeutics, and other bioactive agents to heart and other tissue.

### BACKGROUND

Coronary artery disease (CAD)-mediated heart failure remains one of the leading causes of death and disability in America. The occluded coronary artery impairs blood supply to heart muscle and gradually leads to severe consequences including ischemic heart attack, myocardial infarction, and congestive heart failure. The efficacy of intravenous, intracoronary, and intramyocardial injection of human stem cells to restore heart functions has been attempted, however, the quick loss of injected cells in the beating and ischemic environment significantly limits clinical outcomes.

For example, others have utilized phosphate buffered saline (PBS) to deliver cells to the heart. Cells in PBS are quickly washed out of the heart due to the lack of a bulk material to retain the cells in tissue. People have therefore attempted to utilize other natural materials, such as collagen, fibrin, and chitosan, to embed cells. Others have also attempted to use decellularized animal tissue to embed and deliver cells to the heart in a similar way to injections. However, the immune response towards these materials and their quick degradation can lead to significant issues after being implanted in the heart. Synthetic injectable polymers for delivering cells have also experienced similar problems.

Hence, there remains a need for compositions and methods for delivering cells, therapeutics, and other agents to tissue, and particularly heart tissue. Such compositions and methods should achieve a sustained release of cells and therapeutics, be biodegradable and biocompatible, and encapsulate cells for extended time periods without unduly affecting cell viability.

### BRIEF DESCRIPTION OF THE DRAWINGS

FIG. 1 shows an example of an injectable polymer microgel of the present invention and in vivo peptide release. A) A co-polymer of 21% polyethylene glycol (PEG) and 79% poly-ε-caprolactone (PCL) (%: molar ratio) was synthesized by ring opening polymerization with tin (II) ethyl hexanoate (Sn(Oct)2) catalyst. B) The injectable polymer microgel was a liquid at room temperature (25 C, left) and formed a stable gel at body temperature (37 C, right). C) Injectable polymer microgel immediately after surgery (day 0, left), or after 14 days (right). D) FITC-tagged Ac-SDKP was mixed with polymer microgel at 25 C before injecting either a single bulk 10 mL injection (right), or ten individual 1 mL injections into the muscle around the site of femoral artery ligations. After 7 days, the skin was removed from the thigh muscle and imaged using a fluorescence in vivo imaging system (IVIS). Areas with high fluorescent intensity are colored yellowed in the images. E) Fluorescence intensity of peptides that remained in the tissue after 7 days was quantified using IVIS software. As evidenced by the lower fluorescence intensity of peptides retained in the tissue, more of the peptides were released with multiple 1 mL injections than with the bulk injection over the course of 7 days. De E) n=4; Data are means±SEM from four or five mice. *p<0.05 vs. 1 mL polymer microgel injections (Tukeys' Range Test). (For interpretation of the references to color in this figure legend, the reader is referred to the web version of this article.)

FIG. 2 shows macrophage infiltration with examples of injectable microgels of the present invention in ischemic muscle. Sections of adductor muscle tissue adjacent to peptide-loaded injectable polymer microgels were stained with hematoxylin and eosin (H&E, Ae F) or rat anti-mouse biotinylated F4/80 antibodies (a macrophage marker), as visualized by brown color in images (Ge I). Nuclei were counterstained blue with hemalum. Scale bar=100 mm. Black rectangles indicate magnified area. Black arrows indicate inflammatory infiltration. Yellow arrows indicate blood vessels. Bulk 10 μL injection of microgels without peptide resulted in a high level in inflammatory infiltration (A, G), which was decreased with the co-treatment of C16 and Ac-SDKP peptides (B, H). Multiple 1 μL microgel injections (C) decreased the inflammatory response compared to the bulk injection (A). When delivered with multiple microgel injections, anti-inflammatory Ac-SDKP (D) minimized the inflammatory response, while C16 (E) increased the inflammatory response. The co-treatment (F, I) prevented inflammatory exacerbation while still forming blood vessels. (J) F4/80 staining was quantified by calculating the area of positively stained pixels divided by the total number of cells per image as measured by hematoxylin nuclear stain. Data are means±SEM from four mice per condition. *p<0.05 vs. bulk injection with no peptide; †p<0.05 vs. groups connected by lines (Tukeys' Range Test).

FIG. 3 shows laser Doppler Perfusion Imaging (LDPI) of perfusion recovery from injection of peptide-loaded microgels of the present invention. A) LDPI images of ischemic (right) and un-operated control (left) hind limbs at day 14 after femoral artery ligation and polymer injection with or without peptide loading. B) Perfusion was quantified as the ratio of right to left foot at each time point. Dashed lines represent a single bulk (10 μL) microgel injection, solid lines represent ten, individual 1 μL microgel injections. Data are means±SEM from six mice per condition. *p<0.05 vs. no peptide treatment with microgels at day 14 (Tukeys' Range Test).

FIG. 4 shows angiogenesis and phagocytic activity in injectable microgels. A) Maximum intensity projections of 3D speckle-variance OCT scans were taken of the mouse calf muscle at day 14 after femoral artery ligation and ten, 1 μL intramuscular injections of peptide-polymer microgels around the site of femoral artery ligation. Scale bar=1 mm. B) Blood vessel formation (red) and macrophages phagocytosing E. coli particles (yellow-green) were visualized in tissue with peptide-loaded injectable polymer microgels. Scale bar=50 mm. C) Vessel perfusion capacity as measured by red fluorescence intensity of perfused microspheres extracted from microgels. D) Phagocytic activity (fluorescence intensity per image field). (C,D) Data are means±SEM from four-six mice per condition. *p<0.05 vs. no peptide treatment in same group (Bulk injection or 1 mL injections of microgels); *p<0.05 vs. bulk injection with no peptide; yp<0.05 vs. groups connected by lines (Tukeys' Range Test).

FIG. 5 shows in vitro evaluation of angiogenesis and inflammation by gene expression. Mouse aortic endothelial cells (mAECs) or RAW 264.7 macrophages were cultured with 75 mg/mL of Ac-SDKP, C16, or the combination of C16 and Ac-SDKP peptides for 72 h before isolating RNA and quantitative RT-PCR investigation. Gene expression of matrix metalloproteinase-9 (MMP-9) (A,D), tissue inhibitor-1 (TIMP-1), an inhibitor of MMP-9 (B, E), and tumor necrosis factor-α (TNF-α) (C, F) in mAECs (A, B, C) and RAW 264.7 macrophages (D, E, F). Expression was normalized to GAPDH. Data are means±SEM from eight replicate experiments. (n=8) *p<0.05 vs. no peptide; †p<0.05 between groups connected by lines (Tukeys' Range Test).

FIG. 6 shows macrophage response to TNF-α inhibition. RAW 264.7 macrophages were cultured with 75 mg/mL of Ac-SDKP, C16, or the combination of C16 and Ac-SDKP peptides for 72 h with TNF-α antibodies as soluble TNF-α inhibitors (5 mg/mL). A) TNF-a activity in cell culture supernatants was measured by ELISA. B) MMP-9 activity was measured by zymography. C) MMP-9 activity was quantified by densitometry using Image J and normalized to the average activity of no peptide/no inhibitor treatment condition. Data are means±SEM from four replicate experiments. *p<0.05 vs. no peptide in the same condition (no inhibition or TNF-α inhibition); †p<0.05 between groups connected by lines; ‡<0.05 vs. no inhibition with same peptide treatment (Tukeys' Range Test).

FIG. 7 shows phagocytic activity of RAW 264.7 macrophages with TNF-α inhibition. A) RAW 264.7 macrophages were cultured with peptides (75 mg/mL) and TNF-α antibodies (5 mg/mL) as an inhibitor of TNF-α for 72 h. Images representative of phagocytic activity of macrophages after incubating with green fluorescent E. coli particles (n=4). Scale bar=100 mm. B) Phagocytic activity was quantified by the green fluorescence intensity. Data are means±SEM. *p<0.05 vs. no peptide treatment in same condition (no inhibition or TNF-a inhibition); †p<0.05 between groups connected by lines; ‡p<0.05 vs. same peptide treatment without inhibitor (n=4; Tukeys' Range Test).

FIG. 8 shows mAECs response to TNF-α and MMP-9 inhibition. Ae B) Mouse aortic endothelial cells (mAECs) were cultured with 75 mg/mL of Ac-SDKP, C16, or the combination of C16 and Ac-SDKP peptides for 72 h with MMP-9 inhibitors (5 mM) or TNF-α antibodies as soluble TNF-α inhibitors (5 mg/mL). A) MMP-9 activity was measured by zymography. B) MMP-9 activity was quantified by densitometry using Image J and normalized to the average activity of no peptide/no inhibitor treatment. C) mAECs were cultured on growth factor reduced Matrigel for 6 h before imaging tube formation. Images are representative of 4 replicate experiments. Scale bar=100 mm. Data are means±SEM. *p<0.05 vs. no peptide in same condition (no inhibition or TNF-α inhibition); †p<0.05 between groups connected by lines; ‡p<0.05 vs. no inhibition with the same peptide treatment (Tukeys' Range Test).

FIG. 9 shows PEG-PCL copolymer (a) dissolved in PBS at RT and (b) gelled at 37° C. within 5 min (Sol-gel transition). Collagen-binding study was performed by adding peptide-modified polymer to collagen-coated surface at 37° C., and unbound polymer was washed away. Dylight488-conjugated antibodies targeting PEG was used to recognize (c) the adhered modified polymer (green fluoresence) (d) unmodified polymer (no fluorescence). (e) FACS analysis of induced cardiomyocytes using cardiac makers cTnT, RYR2, and α-actinin. (f) The iPSC-CMs were further verified by immunostaining of cTnT and α-actinin. The encapsulation study was performed by mixing cells with polymer solution and gelled at 37° C. After two weeks, live cells were stained by calcein AM (green fluorescence) (g). Cardiac marker expression was assessed by PCR (h). Column 1: cardiomyocyte positive control; 2: iPSC-CMs encapsulated in hydrogel for 2 weeks; 3: iPSC-CMs grown atop polymer hydrogels in tissue culture plates for 2 weeks. Scale bars represent 100 μm.

FIG. 10 shows the effects of hydrogel-encapsulated iPSC-CMs of the present invention on left ventricle function and remodeling. (a-c) Echocardiography was performed before the ligation of rat LAD coronary arteries (baseline) and again 2 weeks post-delivery (n=5 rats per group). (a, b) The decline in left ventricular (LV) fractional shortening (FS) was significantly less in iPSC-CMs plus polymer (cells+polymer) group compared to other groups (one-way ANOVA, * p<0.05 vs. PBS and cells only groups, t p<0.01 vs. polymer only group). (c) There was a trend toward less LV enlargement in cells plus polymer group compared to other groups, reaching *p<0.05 vs. cell only group. (d) LV wall thickness of the cells plus polymer group was significantly higher than all other groups based on histology staining (one-way ANOVA, †p<0.001 vs. sham, cells only and polymer only groups; n=3 per group). (e) H&E staining of rat hearts. (f) Staining for human nuclei around the infarct area in rat myocardum two weeks after MI/transplantation demonstrated the presence of human nuclei in the heart injected with human iPSC-CMs within polymer hydrogel (arrows). Implanted cells were cardiac α-actinin positive at 2-weeks post-delivery (brownish, bottom right). Scale bars: 50 μm.

### DESCRIPTION

The details of one or more embodiments of the presently-disclosed subject matter are set forth in this document. Modifications to embodiments described in this document, and other embodiments, will be evident to those of ordinary skill in the art after a study of the information provided in this document. The information provided in this document, and particularly the specific details of the described exemplary embodiments, is provided primarily for clearness of understanding and no unnecessary limitations are to be understood therefrom. In case of conflict, the specification of this document, including definitions, will control.

The presently-disclosed subject matter includes compositions that comprise a temperature-responsive polymer and one or more bioactive agents (e.g., cells and therapeutics). Embodiments of the temperature-responsive (thermo-responsive) polymers include injectable polymers which are soluble in aqueous solutions at room temperature so that they can be mixed with one or more bioactive agents. Such polymers undergo a solution-to-gel transition at a transition temperature (Ts). In some embodiments the composition comprises a copolymer, and in certain embodiments the copolymer is a monomethoxypoly(ethylene glycol)-co-poly(ε-caprolactone) (mPEG-PCL) copolymer.

In some embodiments the transition temperature of a composition can be tuned to a predetermined temperature by varying the relative concentration of components within a composition. For example, for compositions comprising a mPEG-PCL copolymer, the transition temperature of the composition can be tuned by adjusting the relative concentrations of mPEG and PCL. In some embodiments the transition temperature is about 20° C., 25° C., 30° C., 35° C., 40° C., 45° C., or 50° C. In certain embodiments the transition temperature is about body temperature (i.e., about 37° C.).

Other embodiments include a microgel system that employs a peptide combination to achieve the dual therapeutic effects, promoting angiogenesis and minimizing inflammatory responses in an uncoupled fashion. “As used herein, the term microgel is a gel formed from a network of filaments of polymer.”

Exemplary compositions can further comprise one or more cells or bioactive agents (e.g., therapeutics). The type of cells are not particularly limited, but can be stem cells in some embodiments. For instance, the cells may include human induced-pluripotent stem cells (iPSC)-derived cardiomyocyes. Furthermore, the term “bioactive agent” as used herein refers to any compound or entity that alters, promotes, speeds, prolongs, inhibits, activates, or otherwise affect biological or chemical events in a subject. Bioactive agents may include, but are not limited, anti-HIV substances, anti-cancer substances, antibiotics, immunosuppressants, anti-viral agents, enzyme inhibitors, neurotoxins, opioids, hypnotics, anti-histamines, lubricants, tranquilizers, anti-convulsants, muscle relaxants, anti-Parkinson agents, anti-spasmodics and muscle contractants including channel blockers, miotics and anti-cholinergics, anti-glaucoma compounds, anti-parasite agents, anti-protozoal agents, and/or anti-fungal agents, modulators of cell-extracellular matrix interactions including cell growth inhibitors and anti-adhesion molecules, vasodilating agents, inhibitors of DNA, RNA, or protein synthesis, anti-hypertensives, analgesics, anti-pyretics, steroidal and non-steroidal anti-inflammatory agents, anti-angiogenic factors, angiogenic factors, anti-secretory factors, anticoagulants and/or antithrombotic agents, local anesthetics, ophthalmics, prostaglandins, anti-depressants, anti-psychotics, targeting agents, chemotactic factors, receptors, neurotransmitters, proteins, cell response modifiers, cells, peptides, polynucleotides, viruses, and vaccines. In certain embodiments, the bioactive agent is a drug and/or a small molecule.

In some embodiments the composition is conjugated to a peptide. The peptide can be a functional peptides that enhances the ability of the composition to adhere to fibrous tissue. In specific embodiments the functional polypeptide is a decorin-derived peptide, which can bind to collagenous tissue to make the composition self-adhesive onto fibrotic tissues in vivo. The adhesion enhancement due to the presence of a functional peptide can be desirable for cell integration and tissue regeneration. Indeed, fibrotic tissues are usually formed at a site of injury and/or ischemia with inflammatory responses.

The compositions described herein therefore have the superior and unexpected advantage of having tunable transition temperatures and of being biodegradable and biocompatible. In this regard, the term “biodegradable” as used herein generally refers to materials that degrade under physiological conditions to form a product that can be metabolized or excreted without damage to the subject. In certain embodiments, the product is metabolized or excreted without permanent damage to the subject. Biodegradable materials may be hydrolytically degradable, may require cellular and/or enzymatic action to fully degrade, or both. Biodegradable materials also include materials that are broken down within cells. Degradation may occur by hydrolysis, oxidation, enzymatic processes, phagocytosis, or other processes.

The term “biocompatible” as used herein generally refers to materials that, upon administration in vivo, do not induce undesirable side effects. In some embodiments, the material does not induce irreversible, undesirable side effects. In certain embodiments, a material is biocompatible if it does not induce long term undesirable side effects.

The presently-disclosed subject matter further relates to methods of treating tissue in a subject, such as heart tissue, by administering an effective amount of the present composition to the subject. The compositions may be administered by injection. The compositions and methods therefore provide a minimally invasive way to deliver cells and/or other bioactive agents to tissue for tissue regeneration.

The term “subject” refers to a target of administration, which optionally displays symptoms related to a particular disease, pathological condition, disorder, or the like. The subject of the herein disclosed methods can be a vertebrate, such as a mammal, a fish, a bird, a reptile, or an amphibian. Thus, the subject of the herein disclosed methods can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. A patient refers to a subject afflicted with a disease or disorder. The term “subject” includes human and veterinary subjects.

Embodiments of compositions that adhere to collagenous tissue (e.g., scar tissue, fibrotic tissue) can permit the delivered components (e.g., cells and bioactive agents) to be retained in the tissue for a relatively long period of time. In some embodiments the delivered components can be retained in a viable state for about 1 day, 5 days, 10 days, 15 days, 20 days, 25 days, 30 days, or longer. Thus, for example, delivered cells can be retained in a viable state within the composition until the cells migrate and integrate into the tissue. This can be particularly desirable for the extended delivery and regeneration of wounded or otherwise damaged tissue.

Some embodiments of compositions and methods are for treating heart tissue. This includes ischemic or otherwise damaged heart tissue. Causes for heart tissue damage include myocardial infarction. Thus, embodiments of methods can regenerate wounded heart tissue by promoting angiogenesis in the damaged heart tissue.

Thus, one embodiment of the present invention is a polymeric compound a co-polymer microgel at least one bioactive agent. In other embodiments, the microgel may comprise monomethoxypoly(ethylene glycol)-co-poly(ε-caprolactone) (mPEG-PCL). In other embodiments, the mPEG is about 750 Da. In other embodiments, the microgel comprises about 21% PEG-b-79% PCL.

Examples of the bioactive agent include at least one pro-angiogenic peptide and at least one anti-inflammatory peptide. In embodiments of the invention, the pro-angiogenic peptide is C16 (Lys-Ala-Phe-Asp-Ile-Thr-Tyr-Val-Arg-Leu-Lys-Phe) and the anti-inflammatory peptide is Ac-SDKP (N-acetyl-Ser-Asp-Lys-Pro).

In other examples, the bioactive agent selectively binds to fibrous tissue. In yet other examples, the bioactive agent includes a decorin-derived functional peptide. In others, bioactive agent is at least one cell, optionally including stem cells.

In other examples, the microgel comprises a co-polymer of the following formula:

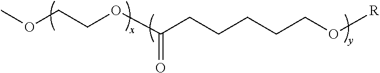

wherein x is 10-50% and y is 90-50%, and R is the bioactive agent.

In embodiments of the present invention, the composition includes a transition temperature of about 20° C. to about 50° C. In other embodiments, transition temperature is about mammalian body temperature.

Another embodiment of the present invention is a composition comprising a polymeric compound described herein and a pharmaceutically acceptable carrier.

Another embodiment of the present invention is a method for treating damaged tissue to a patient in need thereof, comprising providing a composition of the present invention, and administering by injection an effect amount of the composition.

The following is an example of a synthetic scheme of mPEG-PCL and the modification with collagen-binding peptide.

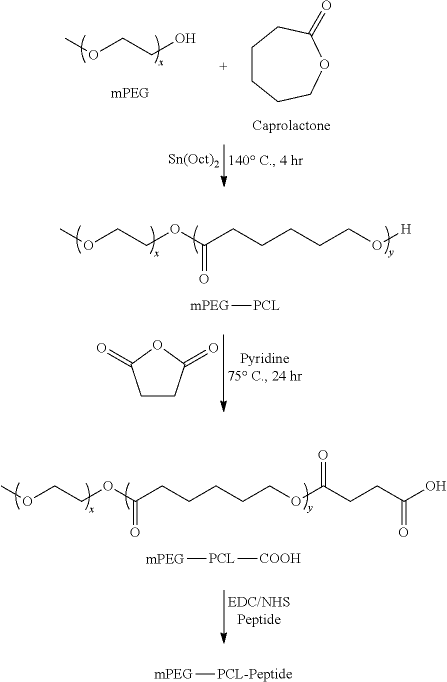

### EXAMPLES

The presently-disclosed subject matter is further illustrated by the following specific but non-limiting examples. The following examples may include compilations of data are representative of data gathered at various times during the course of development and experimentation related to the presently-disclosed subject matter.

While the terms used herein are believed to be well understood by one of ordinary skill in the art, the definitions set forth herein are provided to facilitate explanation of the presently-disclosed subject matter.

Unless defined otherwise, all technical and scientific terms used herein have the same meaning as commonly understood by one of ordinary skill in the art to which the presently-disclosed subject matter belongs. Although any methods, devices, and materials similar or equivalent to those described herein can be used in the practice or testing of the presently-disclosed subject matter, representative methods, devices, and materials are now described.

Following long-standing patent law convention, the terms “a”, “an”, and “the” refer to “one or more” when used in this application, including the claims. Thus, for example, reference to “a composition” includes a plurality of such compositions, and so forth.

Unless otherwise indicated, all numbers expressing quantities of ingredients, properties such as reaction conditions, and so forth used in the specification and claims are to be understood as being modified in all instances by the term “about”. Accordingly, unless indicated to the contrary, the numerical parameters set forth in this specification and claims are approximations that can vary depending upon the desired properties sought to be obtained by the presently-disclosed subject matter.

As used herein, the term “about,” when referring to a value or to an amount of mass, weight, time, volume, concentration or percentage is meant to encompass variations of in some embodiments ±50%, in some embodiments ±40%, in some embodiments ±30%, in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed method.

As used herein, ranges can be expressed as from “about” one particular value, and/or to “about” another particular value. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

### Example 1

This Example demonstrates microgels of the present invention from a combination of polyethylene glycol (PEG) and poly-ε-caprolactone (PCL), and embodiments of the present invention where the polymer microgels of the present invention are C16 and/or Ac-SDKP loaded and injected to increase collateral vessel formation without inflammatory exacerbation. This example is also described in Zachman et al., Biomaterials 35 (2014) 9635-9648, the contents of which are incorporated herein by reference.

Materials and Methods

Chemicals and reagents for injectable polymer microgel: Tin (II) ethyl hexanoate (Sn(Oct)2), ε-caprolactone, monomethoxypoly(ethylene glycol) (mPEG) (Mₙ=750 Da), anhydrous tetrahydrofuran (THF), anhydrous toluene, dichloromethane, and diethyl ether were purchased from Sigmae Aldrich (St. Louis, Mo., USA). ε-Caprolactone was dried and distilled over CaH2 (Alfa Aesar, Ward Hill, Mass., USA) immediately before polymerization. Tin (II) ethyl hexanoate was distilled under high vacuum.

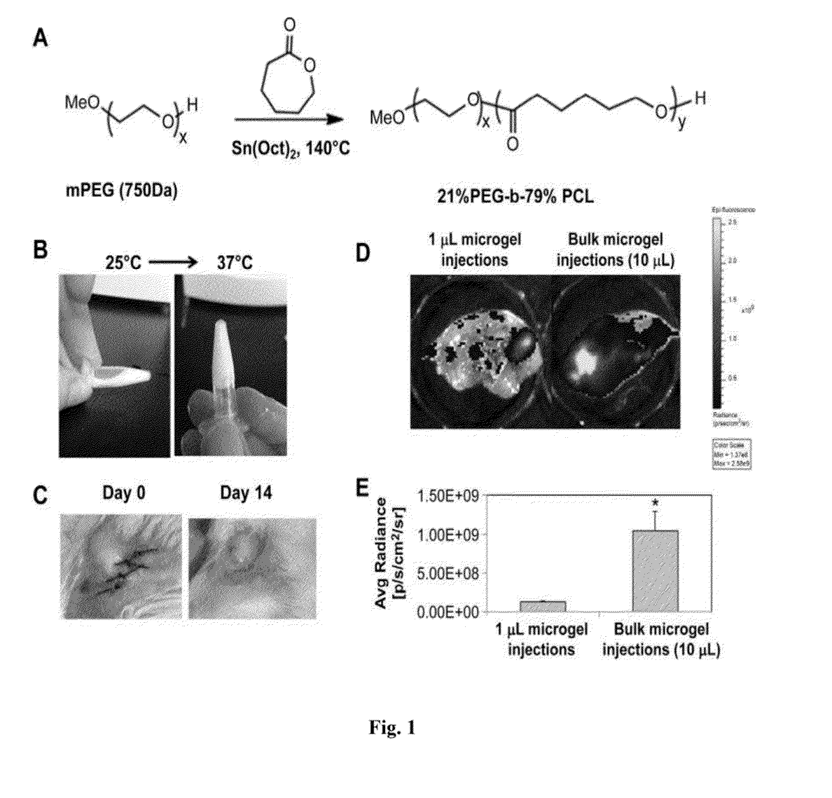

Synthesis and characterization of injectable polymer microgels: The injectable polymer is presented as 21% PEGe 79% PCL (individual mole percentage) (FIG. 1A). Our previous studies have showed that this format of combinatorial polymers provides tunable degradation, mechanical, and thermal properties by changing the mole percentages. PEGe PCL was synthesized by ring opening polymerization of 8-caprolactone according to previously published methods. The structure and the number average molecular weight (Mₙ) calculated by the molar ratio of PEG and PCL was verified by NMR. Injectable polymers were dissolved in H₂O to form a 13% polymer by weight solution at 25 C.° and then incubated at 37° C. and observed every 10 s until a stable gel formed to determine the gelation time (FIG. 1B).

In vitro biocompatibility assay: HUVECs (ATCC) were seeded at a density of 1×10⁵ cells/mL in MesoEndo media (Cell Applications) on top of pre-gelled injectable polymer microgels and cultured for 1 or 3 days at 37 C with 5% CO2 and stained with LIVE/DEAD® Viability/Cytotoxicity Kit (Invitrogen) according to the supplier's protocol (n=4 per condition).

Mouse model of hind limb ischemia: Wild type A/J mice were used to develop a model of PAD as described previously, by ligating the femoral artery and vein at one ligation below the epigastric artery and a second ligation around the artery and vein at a distal location just proximal to the deep femoral branch. The femoral artery and vein were then cut between these two sutures. A 13% by weight solution of injectable polymers in H2O 2O was mixed with 75 mg Ac-SDKP, C16, or the combination of Ac-SDKP and C16 at 25° C. In order to control the hydrogel size considering the possibility that the hydrogel size may change peptide release and therefore inflammatory responses a single, 10 mL bulk injection or ten, 1 mL injections of peptide-loaded polymer were made into the thigh muscle adjacent to the femoral artery ligations. The surgical incision was then closed with non-degradable sutures. As controls, femoral artery ligation surgery was performed on animals without any microgels or peptide treatment or with peptide in PBS injections into the subcutaneous tissue adjacent to femoral artery ligations. The left hind limb (unoperated) was also used as a surgical control.

In vivo peptide release from injectable microgels: A 13% by weight solution of injectable polymers in H2O 2O was mixed with 75 mg of FITC-labeled SDKP (GenScript) at 25 C. A single, 10 mL bulk injection or ten, 1 mL injections of peptide-loaded polymer were made into the thigh muscle adjacent to the femoral artery ligations. After 7 days, mice were sacrificed by CO2 inhalation and death was verified by cervical dislocation. The skin on the ischemic hind limb was removed and the adductor muscle was imaged on an IVIS 200 pre-clinical in vivo imaging system (Perkin Elmer, Waltham, Mass.) to visualize peptide retention in the tissue (n=4 mice per treatment).

Non-Invasive Imaging of Ischemia

LDPI: LDPI was performed on the footpad region of the hind limb of the mice using a Periscan PIM II device. This technique images surface perfusion by measuring Doppler changes in the reflectance of light due to blood flow. During imaging, ambient light and temperature were carefully controlled to avoid background variations in LDPI measurements. Three scans were performed per mouse at each time point: days 0, 3, 7 and 14 after femoral artery ligation and microgel injection (n=6 mice per treatment). The perfusion ratio was calculated by normalizing the average perfusion value of the ischemic footpad (right) to the average perfusion value of the control, un-operated footpad (left) using Image J (NIH).

Optical coherence tomography: Doppler OCT and speckle-variance OCT were used to non-invasively image blood vessels in the ischemic gastrocnemius muscle of mice on days 1 and 13 after femoral artery ligation, as previously described. Doppler OCT detects frequency shifts in the phase-sensitive OCT signal due to flowing blood, while speckle-variance OCT tracks variation in laser speckle over time due to red blood cell movement. Doppler OCT cross-sectional scans (B-scans) were used to quantify blood flow changes over time in the hind limb, while volume intensity projections from speckle-variance OCT image volumes (C-scans) presented vessel morphology differences between groups. The OCT system uses an 860 nm center wavelength, 51 nm bandwidth laser, and has an axial resolution of 6.4 mm in air and lateral resolution of 25 mm. Prior to imaging, mice were anesthetized and hair on the hind limb was removed. To track the imaged area over time, glass microscope slides were marked with the placement of the mouse during imaging day 1 and used to correctly position the mouse leg during imaging on day 13. To avoid bulk motion artifacts, OCT scans of the calf muscle were gated between breaths of the mouse. Six Doppler OCT B-scans (4 mm, 800 A-scans, Doppler number of 9) were performed per mouse at each time point and perfusion was quantified by calculating the ratio of the number of blood vessel pixels per scan over the total imaged area per scan (n=6 mice per time point).

Angiogenesis and phagocytosis assays: Fourteen days after femoral artery ligation, mice were sacrificed and tissue and microgels were harvested for analysis. Immediately before sacrificing the mice, functional fluorescence micro-angiography was performed to visualize angiogenesis in and around peptide-loaded microgels, as described previously. As a result of fluorescence microangiography, only the functional capillaries with a perfusion capacity, including those around injected micro-gels, show red fluorescence in the mouse body. Excised tissue from the site of microgel injections was imaged using an Olympus FV100 confocal microscope. For quantification of vessel perfusion capacity, the red fluorescence intensity was quantified using Image J software (n=6 images per mouse, n=6 mice per treatment).

A phagocytosis assay was performed in harvested microgels using Vybrant Phagocytosis Assay kit according to the manufacturer's protocol. Green fluorescence from internalized Escherichia coli particles in excised microgels and surrounding tissue was visualized through confocal imaging. The intensity of green fluorescence in each image was quantified using Image J (n=6 images per mouse, n=6 mice per treatment).

Histological analysis of angiogenesis and inflammation: After sacrificing mice, ischemic muscle samples were prepared for histological analysis as described elsewhere. Briefly, hind limbs were detached and placed in methanol overnight after removal of skin. Adductor muscle samples adjacent to the micro-gels were cut from the limb and placed in 10% phosphate buffered formalin for 24 h, embedded in paraffin, sectioned (5 mm sections), mounted on slides, antigen retrieval, and stained with either he-matoxylin and eosin (H&E), or biotinylated rat anti-mouse F4/80 antibodies by the Vanderbilt Translational Pathology Shared Resource Core. Immunohistochemical (IHC) staining to identify activated inflammatory cells by F4/80 expression was quantified by normalizing the total F4/80 positive area (indicated by brown staining) to the total cell number (determined by hema-toxylin nuclear staining) using Image J.

Cell Culture: RAW 264.7 macrophages (ATCC) were cultured in DMEM (Gibco) supplemented with 10% FBS and 1% penicillin/strepto-mycin. Mouse aortic endothelial cells (mAECs) were a generous gift from Dr. Ambra Pozzi at Vanderbilt University Medical Center. mAECs were cultured in EGM-2 Basal Media supplemented with BulletKit (Lonza, Allendale, N.J.) and 10 units/mLIFN-g (Sigma). RAW 264.7 mouse macrophage cells (Sigma) were cultured in DMEM with 10% FBS and 1% penicillin/streptomycin. For cell culture studies with peptides, 75 mg/mL of Ac-SDKP or C16 peptides was used. For inhibition studies, 5 mM of MMP-9 inhibitor-1 (CTK8G1150; AG-L-66085, Santa Cruz Biotechnology, Dallas, Tex.) or 5 mg/mL of LEAF Purified Mouse TNF-a antibody (BioLegend) was used.

In vitro peptide uptake: mAECs or RAW 264.7 cells were incubated with DilC12 (BD Biosciences) for 2 h; washed two times with PBS; and seeded 3×10⁵ cells/mL on pre-gelled injectable polymer microgels loaded with FITC-tagged Ac-SDKP or C16 peptides (75 mg peptide/mL media, GenScript). After 72 h, cells were washed with PBS and imaged using Zeiss LSM 710 confocal microscope for visualization of peptide uptake (n=4 per treatment).

Gene expression: mAECs or RAW 264.7 cells were seeded at a density of 3×10⁵ cells/mL on TCPS with 75 mg/mL of Ac-SDKP, C16, or the combination of C16 and Ac-SDKP peptides. After 3 days, RNA was extracted from homogenized tissue using Trizol reagent and RNA easy columns. After dissolving RNA in RNase-free water, the concentration and purity of isolated RNA was measured using TECAN plate reader Nanoquant (company info). At least 1.2 mg of RNA was reverse transcribed using iScriptReverse transcription Supermix for RT-qPCR on a BioRad thermocycler (company info). 50 ng/well cDNA was then amplified using SYBR green Supermix and fluorescence signal was measured on a BioRAD real time PCR machine. TGF-b1 forward: GCTGAACCAAGGAGACGGAA, reverse: AGAAGTTGGCATGGTAGCCC. NF-kb forward: ATGTAGTTGCCACG-CACAGA, reverse: GGGGACAGCGACACCTTTTA. TIMP1 forward: AGACACACCAGAGATACCATGA, reverse: GAGGACCTGATCCGTC-CACA. FGF-1 forward: TCTGAAGAGTGGGCGTAGGA, reverse: GGCTATTTGGGGCCATCGTA. FGF-2: MMP-9: TTGAGTCCGGCAGA-CAATCC, reverse: CCTTATCCACGCGAATGACG. MMP-2 forward: GAGTTGGCAGTGCAATACCT, reverse: GCCGTCCTTCTCAAAGTTGT. TNF-a: forward: ACGGCATGGATCTCAAAGAC, reverse: AGA-TAGCAAATCGGCTGACG. VEGF forward: ATGCGGATCAAACCT-CACCA, reverse: CCGCTCTGAACAAGGCTCAC. GAPDH forward: TGAAGCAGGCATCTGAGGG, reverse: CGAAGGTGGAAGAGTGGGAG. TIMP-2 forward: CTCGCTGGACGTTGGAGGAA, reverse: CACGCG-CAAGAACCATCACT. Expression was calculated using the 2 method and normalized to GAPDH expression (n=8).

MMP-9 activity: After 72 h culture in serum-free media, culture media from mAECs or RAW 264.7 cells (3×10⁵ cells/mL, with/without peptides or TNF-a/MMP-9 inhibitors) was collected, concentrated, and analyzed by zymography, as described previously. Concentrated protein samples were incubated with non-reducing buffer at a ratio of 1:1. 9.5 mg total protein per lane were loaded onto a 7.5% polyacrylamide gel containing 0.1%(w/v) gelatin (Sigma). Gels were washed in dH2O containing 3.3% (v/v) Triton X-100, followed by 12e 48 h incubation in reaction buffer at 37 C. After incubation, gels were placed in fixative (30% methanol, 10% acetic acid, and 60% dH2O) for 1 h before staining with 4 parts Coomassie brilliant blue R-250 (Sigma) and 1 part methanol for 12 h. Gels were destained in 25% methanol for 1 h. The gelatinolytic activity of pro-MMP-9 was determined by densitometry of the 97 KDa white band on a blue background using Image J(n=4 per treatment). MMP-9 expression was then normalized to the expression from the no inhibitor, no peptide treated group.

Phagocytic activity: RAW 264.7 cells (3×10⁵ cells/mL) were cultured with Ac-SDKP, C16, or the combination of C16 and Ac-SDKP peptides (75 mg/mL) in the presence or absence of TNF-a inhibitor or MMP-9 inhibitor for 72 h. Vybrant phagocytosis assays were used to evaluate the inflammatory activity, as described above in the in vivo section. (n=4 per treatment).

Tubulogenesis: mAECs (3×10⁵ cells/mL) were cultured on growth factor reduced Matrigel (200 mL, BD Biosciences) for 6 h before imaging for tube formation using a Nikon Eclipse Ti microscope (n=4 per treatment).

ELISA: To verify TNF-a inhibition with antibodies, an ELISA was performed using Mouse TNF-a ELISA MAXTM Deluxe (Biolegend) according to the supplier's protocol. Secreted TNF-a in RAW 264.7 cell culture supernatant was measured by reading absorbance at 450 nm using a TECAN M1000 plate reader and quantified against a standard curve (n=4 per treatment).

Statistics: To determine if statistical significance existed between groups, one-way ANOVA was performed between groups followed by Tukey's range tests for comparisons between groups. For all experiments, p<0.05 was considered statistically significant and results were presented as means±standard error of the mean.

Results

Injectable polymer fabrication and characterization: The co-polymer of 21% PEGe 79% PCL (%: molar ratio) was synthesized by reacting 8-caprolactone with methoxyPEG (mPEG) using tin(II) ethyl hexanoate as a catalyst (FIG. 1A). The number average molecular weight (Mₙ) of resulting polymer was 3571 Da as verified by NMR. The polymer structure was verified by 1H NMR spectra: ¹H NMR (CDCl3)=d 4.06 (t, 3H, —OCH2), 3.65 (s, 4H, —OCH2), 2.31 (t, 2H, —CH2), 1.66 (m, 2H, —CH2), 1.37 (m, 4H, —CH2) ppm. This polymer was dissolved in water and easily mixed with peptides at 25 C. When the temperature increased to 37 C, it underwent successful sole-gel transition and formed a stable gel within 15 s (FIG. 1B).

Cell viability with injectable polymer microgels: To determine the biocompatibility of injectable polymer microgels, human coronary artery endothelial cells (HCAECs) were cultured on pre-gelled polymer microgels for three days. HCAECs maintained viability (green by calcein AM) with few dead cells (red by ethidium homodimer). Of note, some larger areas of red fluorescence are from the polymer itself due to auto-fluorescence, and not necessarily indicative of dead cells.

Peptide uptake by macrophages and endothelial cells: Fluorescein isothiocyanate (FITC)-tagged C16 and Ac-SDKP peptides were used in mouse aortic endothelial cells (mAECs) and macrophages (RAW 264.7 cells) cultures to examine cellular uptake of peptides. Both cell types internalized Ac-SDKP and C16 peptides where C16 was confined in punctuate, distinct areas in the cells, while Ac-SDKP was diffusely present throughout the cells.

In vivo peptide release from injectable polymer microgels: To evaluate the ability of these peptide loaded-microgels to regulate angiogenesis and inflammation in PAD, a mouse model of hind limb ischemia was used. The A/J mouse strain was chosen due to its prolonged time course recovery from hind limb ischemia to mimic the delayed recovery seen in human PAD. When polymer solutions were injected into the muscle at the site of femoral artery ligation, they rapidly formed a stable gel (FIG. 1C). Polymer microgels, mixed with or without peptides, were delivered either by a single, bulk, 10 mL injection or ten individual 1 mL injections into the adductor muscle adjacent to the ligation sites. Even 14 days after injection, the 10 mL bulk microgel was still visible without any significant change in size and gelation status. To measure peptide release from these injectable microgels in vivo, FITC-tagged Ac-SDKP was mixed with polymer solutions before injection. After 7 days, the single 10 mL bulk injection of microgel retained over 10 times more of the peptides than multiple 1 mL injections, indicating faster peptide release from multiple 1 mL injections than the single 10 mL bulk injection (FIG. 1De E).

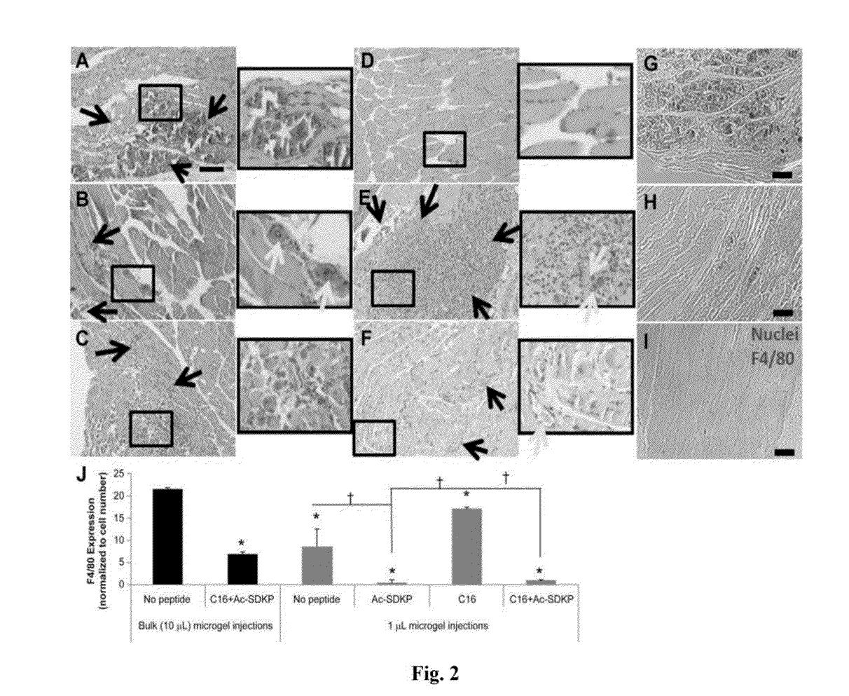

Macrophage recruitment with peptide-loaded injectable polymer microgels: To investigate inflammatory responses to our peptide-loaded microgels at fourteen days after ligation and injection of micro-gels, the adductor muscle tissue adjacent to the microgels was sectioned and stained with hematoxylin and eosin (H&E) (FIG. 2Ae F) and mouse macrophage marker F4/80 (FIG. 2Ge I). Without peptide treatment, bulk injection of microgel resulted in muscle hypertrophy as indicated by varying muscle fiber size, replacement with fibrous and adipose tissues, and inflammatory cell infiltration (FIG. 2A, G); however, multiple 1 mL microgel injections decreased this inflammatory response (FIG. 2C). With multiple 1 mL microgel injections, the incorporation of anti-inflammatory Ac-SDKP peptides (FIG. 2D) further decreased inflammatory infiltration in the muscle tissue, while treatment with pro-angiogenic C16 (FIG. 2E) augmented macrophage infiltration by two fold as compared to no peptide treatment (FIG. 2C). However, co-treatment with C16 and Ac-SDKP successfully regenerated muscle tissue as indicated by centralized nuclei in regenerating muscle fibers in which macrophage infiltration was reduced by 70% in bulk injection (FIG. 2B, H) compared to no peptide treatment (FIG. 2A). The regeneration effect augmented further when co-treated in multiple, low volume microgel injections (FIG. 2F, I).

Even with this co-treatment of the peptides in bulk injection (FIG. 2B, H), the level of macrophage infiltration was still higher than the control PBS injection without microgel or peptides. When multiple 1 mL polymer microgel injections were used (FIG. 21), the level of macrophage infiltration was successfully minimized to a similar level to the control PBS injection without polymer. These results suggest that multiple small volume injections of peptide-loaded microgels are more effective than bulk injection in attenuating the inflammatory response.

The recruitment of inflammatory cells also followed similar trends when the peptide-loaded implantable polymer scaffolds were compared to the injectable polymer microgels (FIG. 2Ce F), with C16 augmenting macrophage infiltration while Ac-SDKP diminished macrophage infiltration. The low level of macrophage infiltration observed under treatment of Ac-SDKP alone (FIG. 2D) was preserved with the co-treatment of C16 and Ac-SDKP (FIG. 2F), confirming the therapeutic ability of these peptides for PAD. Therefore, multiple small volume microgel injections were used to deliver peptides in the following studies unless the injection method was specifically mentioned.

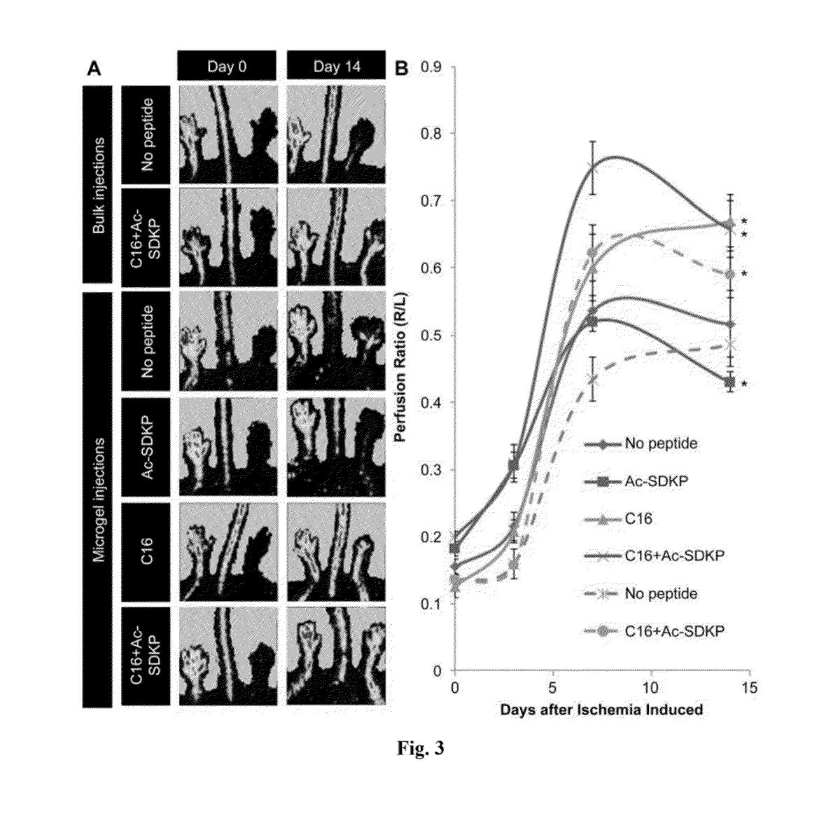

Perfusion recovery with peptide-loaded microgels: Laser Doppler perfusion imaging (LDPI) and optical coherence tomography (OCT) were used to monitor blood perfusion to the ischemic hind limb over the course of 14 days. LDPI was conducted on the foot pads of the mouse hind limbs on 1, 3 7, and 14 days after femoral artery ligation. To quantify perfusion recovery in the ischemic hind limb, the perfusion in the right foot, in which the femoral artery and vein were ligated, was compared to perfusion in the left foot, which was left un-operated as an internal control. Without peptide or microgel treatment, perfusion in the ischemic right foot slightly increased over the course of 14 days, indicating minimal spontaneous recovery of function to the hind limb (FIG. 3 and FIG. S6). However, mice treated with microgels loaded with pro-angiogenic C16 had 29% higher perfusion compared to mice treated with microgels without peptides (FIG. 3). Anti-inflammatory Ac-SDKP loaded microgels did not increase perfusion compared to no peptide treatment (FIG. 3). Without being bound by theory or mechanism, the combination C16 and Ac-SDKP loaded microgels restored perfusion more effectively than any other treatment over the 14 day time course, suggesting a synergistic increase in tissue recovery with the dual peptide treatment (FIG. 3). LDPI also followed similar trends when the peptide-loaded implantable polymer scaffolds were compared to the injectable polymer microgels, with C16 increasing perfusion while Ac-SDKP decreased perfusion. However, the combination of C16 and Ac-SDKP maintained the highest level of perfusion observed. The co-treatment of C16 and Ac-SDKP also preserved the minimal amount of macrophage infiltration, confirming the therapeutic ability of these peptides.

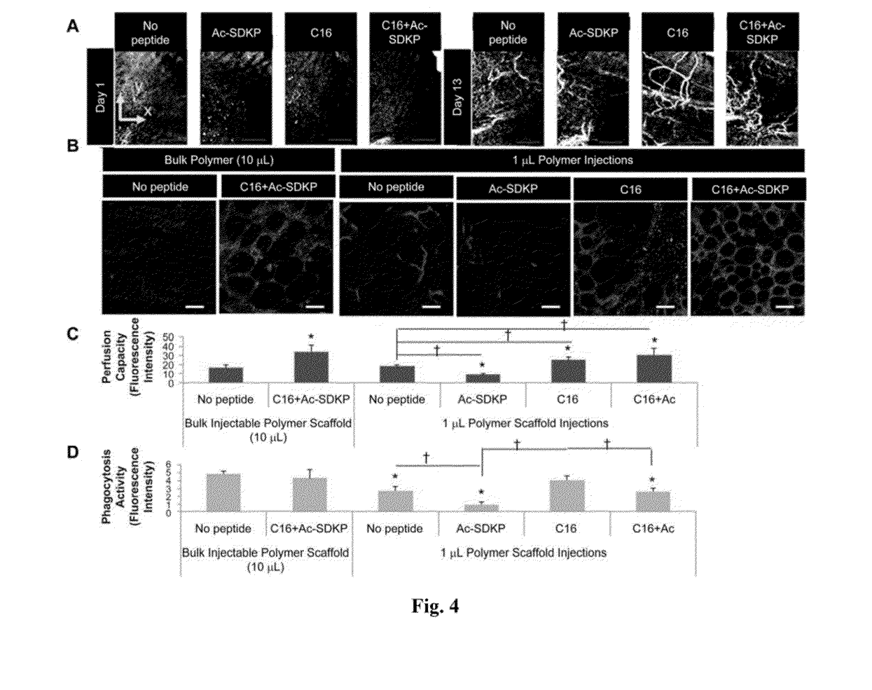

Doppler and speckle-variance OCT were also used to non-invasively image blood flow and vessel morphology in the hind limb, respectively. An increase in both the number and size of blood vessels in the ischemic calf muscle from 1 day to 13 days post-surgery with all treatment conditions was observed in cross-sectional Doppler OCT scans. Quantification of Doppler OCT scans with implantable scaffolds revealed a similar trend to LDPI measurements: treatment with C16 alone or the combination of C16 and Ac-SDKP resulted in a greater than two fold increase in the total area of vessels over the time course compared to treatment with Ac-SDKP or no treatment. No significant difference was observed between C16 alone and the co-treatment of C16 and Ac-SDKP, indicating the ability of this co-treatment to promote blood vessel formation. With injectable microgels, speckle-variance OCT volume scans of the calf muscle revealed Ac-SDKP or no peptide treatment formed few blood vessels with limited branching, while C16 or the combination of C16 and Ac-SDKP formed many vessels with a high degree of branching (FIG. 4A).

Angiogenesis and phagocytosis in peptide-loaded microgels: Fluorescence microangiography and a Vybrant phagocytosis assay were used to quantify angiogenesis and phagocytic activities, respectively in the tissue around microgels at the site of femoral artery ligations. We chose to measure phagocytosis because it is a crucial and potent indicator of inflammatory cell activation. C16-loaded microgels enhanced angiogenesis and macrophage activities in the ischemic hind limb, while Ac-SDKP-loaded micro-gels reduced both responses in comparison to microgels without peptide loading (FIG. 4Be C). Slightly increased angiogenesis was observed upon PBS injections of C16 peptides compared to PBS alone, while microgel-mediated C16 peptide delivery increased angiogenesis almost 1.5 fold vs. microgels without peptides (FIG. 4Be C). Unfortunately, C16 delivered via microgels stimulated the inflammatory response as evidenced by higher phagocytic activity than no peptide controls. However, the incorporation of Ac-SDKP peptides abated this inflammatory response, lessening the phagocytic activity to levels comparable to PBS only. Co-delivery of both Ac-SDKP and C16 peptides increased perfusion capacity 1.7 fold vs. microgels without peptides, while reducing phagocytosis to levels similar to no microgel controls, confirming our previous findings [12].

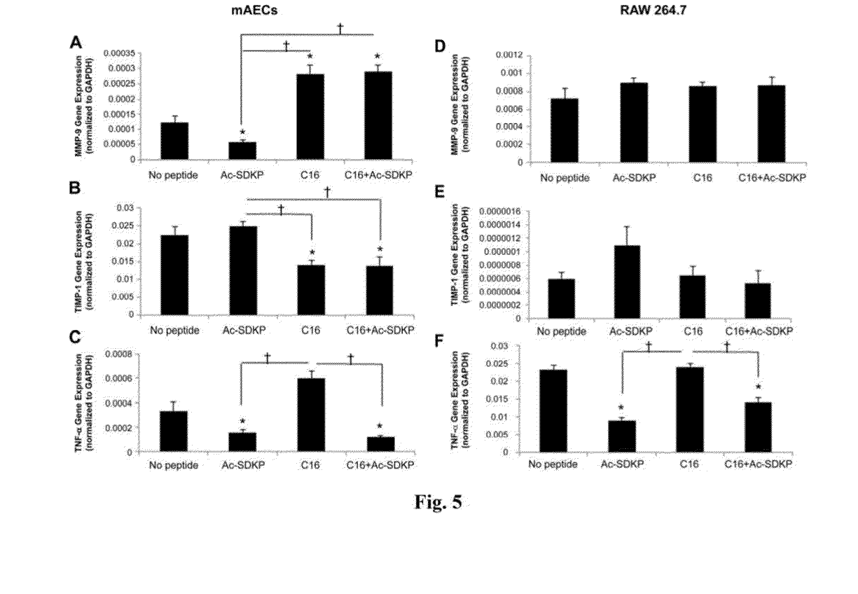

Inflammatory activation, as quantified by a Vybrant phagocy-tosis assay, was highest with bulk injection of microgels, and was attenuated remarkably with multiple, low volume injections of microgels (FIG. 4C). With multiple small volume injections, phagocytosis decreased and perfusion capacity increased, indicating the improved therapeutic efficiency of these peptides when delivered via multiple small volume injections vs. a single bulk injection. 4.8. In vitro evaluation of angiogenesis and inflammation by gene expression Since the therapeutic efficiency of these peptides was demonstrated using injectable microgels in a mouse PAD model, we sought to elucidate a mechanism of uncoupling angiogenesis and inflammation. First, we analyzed expression of angiogenic and inflammatory genes (i.e. MMP-9, TIMP-1, and TNF-a) in mouse aortic endothelial cells (mAECs) and RAW 264.7 macrophages as an in vitro model of angiogenesis and inflammation, respectively (FIG. 5). Compared to no peptide treatment, MMP-9 expression in mAECs increased with C16 or co-treatment (both C16 and Ac-SDKP) over 2-fold, while its expression decreased with Ac-SDKP treatment over 50% (FIG. 5A), following trends similar to angiogenesis in vivo with peptide treatments. The expression of TIMP-1, an inhibitor of MMP-9, showed the opposite trend to MMP-9 expression in response to the peptide treatments in mAECs (FIG. 5B) as C16 and the combination of C16 and Ac-SDKP decreased TIMP-1 expression significantly. In RAW 264.7 macrophages, no significant differences were detected in MMP-9 or TIMP-1 expression, although there were some variations in TIMP-1 expression among the test groups (FIG. 5D, E). The overall expression level of TIMP-1 in RAW 264.7 was negligible when compared to that of mAECs and the overall expression level of MMP-9 in RAW 264.7, suggesting a minute role of TIMP-1 in MMP-9 activation in RAW 264.7 cells. These results illustrate the influence of MMP-9 on angiogenic processes within endothelial cells, while not having a significant effect on inflammatory cells or inflammatory processes in the peptide treatment conditions. However, expression of TNF-a in both mAECs and RAW 264.7 macrophages directly correlated with inflammatory activation as observed in vivo phagocytic activity and macrophage infiltration (FIGS. 2 and 4D). C16 increased TNF-a expression, while Ac-SDKP and the combined peptide treatment decreased TNF-a expression in comparison to no peptide treatment (FIG. 5C, F), suggesting a regulatory role of TNF-a in the in vivo inflammatory response observed from the mouse PAD model.

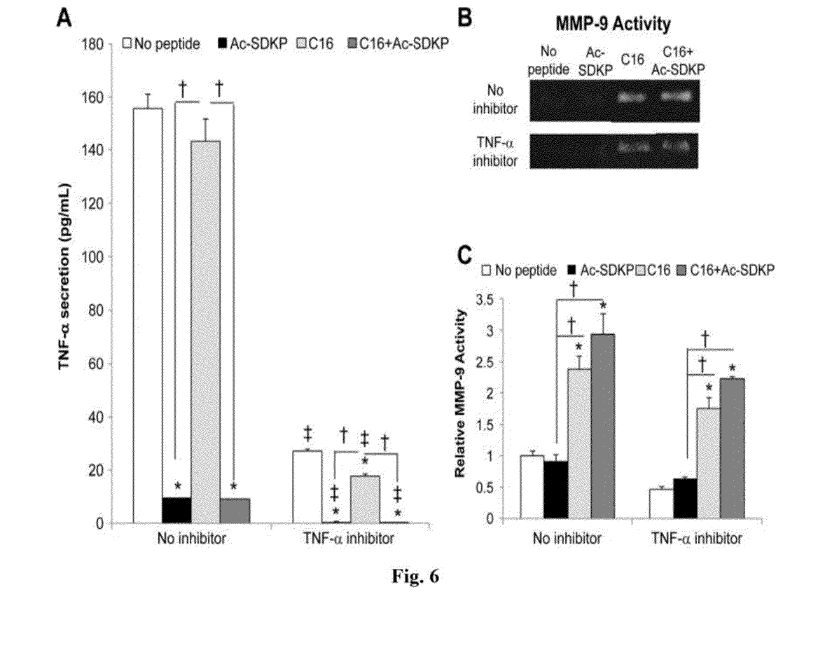

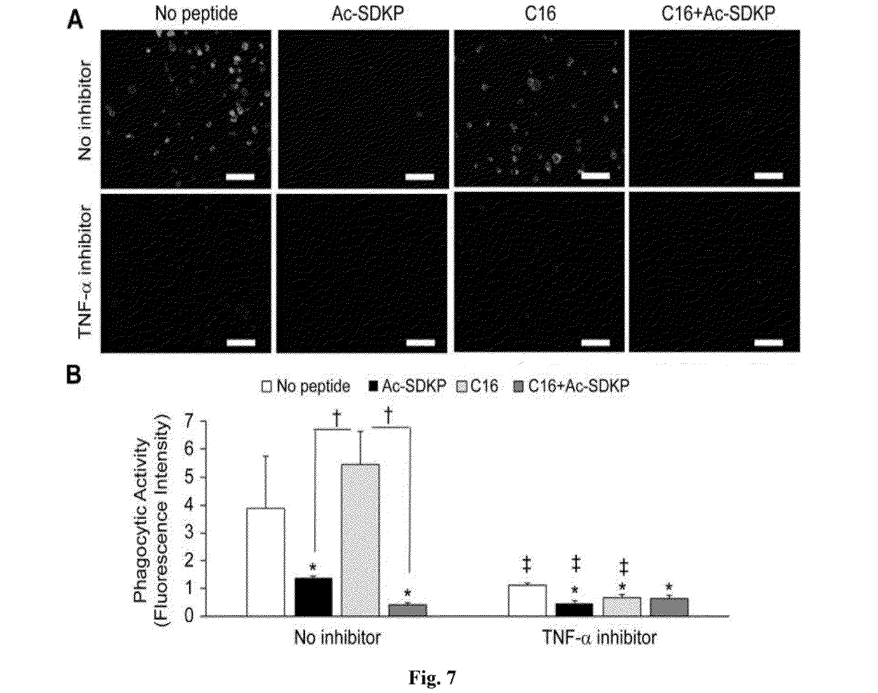

TNF-a and MMP-9 inhibition: To further investigate these mechanisms, inhibitors of TNF-a and MMP-9 were used in cell culture studies. To verify TNF-a inhibition, an ELISA assay was performed. The use of TNF-a antibodies as natural TNF-a inhibitors successfully abrogated cellular production of TNF-a in all the test conditions (FIG. 6A). Regardless of TNF-a inhibitor treatment, Ac-SDKP and the combination peptide-treated macrophages secreted the least amounts of TNF-a (FIG. 6A). To determine if TNF-a influenced MMP-9 activation in response to the peptide treatments, the amount of active MMP-9 produced from macrophages in each condition of peptide treatment was measured by zymography after culturing for 72 h with or without treatment of TNF-a inhibitors. No significant differences were detected in the MMP-9 activities between the no inhibitor and TNF-a inhibitor-treated groups of macrophages in all the test conditions of peptide treatment (FIG. 6Be C). Regardless of TNF-a inhibition, significantly higher levels of MMP-9 activity were observed in macrophages treated with C16 and with the co-treatment of C16 and Ac-SDKP compared to macrophages without peptide treatment. These results indicate that TNF-a inhibition did not influence MMP-9 activity in the peptide treatment conditions. When phagocytic activities were measured using Vybrant phagocytosis assay, TNF-a inhibition minimized the macrophage phagocytic activities in all the test peptide treatment conditions (FIG. 7), confirming that TNF-a is a major regulator of inflammatory responses under peptide treatments (FIG. 5C, F).

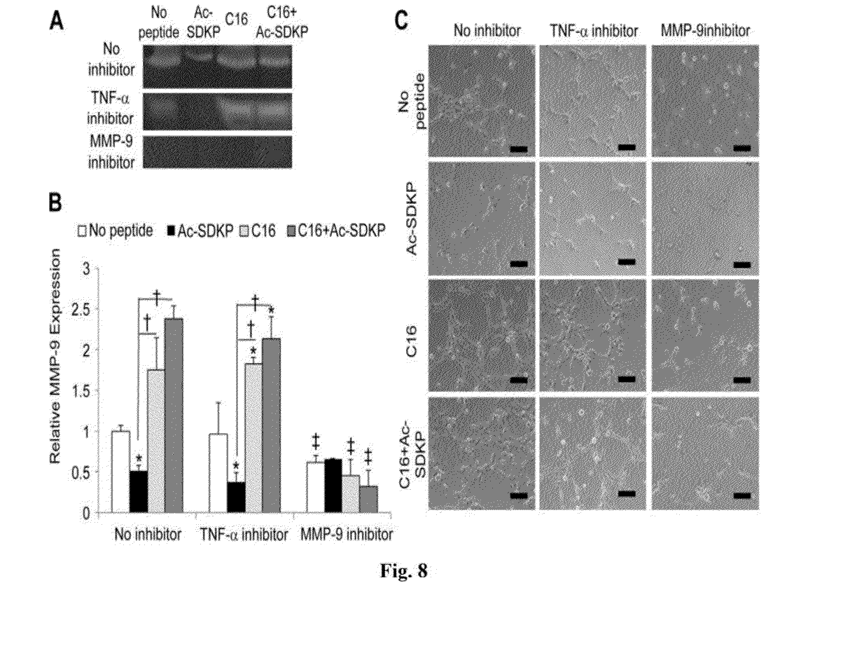

MMP-9 activity was also investigated using a molecular inhibitor of MMP-9 [43]. In endothelial cells, MMP-9 inhibition was verified by the attenuation of MMP-9 activity as measured by zymography (FIG. 8Ae B). While TNF-a inhibition did not significantly influence MMP-9 activity or tubulogenesis in endothelial cells, these activities were significantly reduced when C16 and MMP-9 inhibitor were co-treated (FIG. 8Ae C). Without TNF-a inhibitors, MMP activation and expression, as well as tubulogenesis upon peptide treatments followed similar trends to angiogenesis in vivo. Particularly, C16 or the co-treatment of C16 and Ac-SDKP augmented MMP-9 activity and tubulogenesis, while these effects were diminished with Ac-SDKP treatment (FIG. 8). MMP-9 inhibition reduced these activities significantly to similar levels of Ac-SDKP treatment. These results indicate that angiogenesis was regulated by MMP-9 independently of TNF-a, thereby serving a major mechanism for uncoupling angiogenesis and inflammation under the co-treatment of C16 and Ac-SDKP.

### Discussion of Example 1

The present inventors first demonstrated the new therapeutic effect of combined C16 and Ac-SDKP on PAD when delivered via an injectable polymer scaffold system to mouse hind limb ischemia. This new therapeutic approach promotes angiogenesis while reducing inflammation in a mechanistically uncoupled manner, providing a new idea to the field of PAD therapy with high translational potential.

Although several surgical and non-surgical treatments are available for patients with PAD, there is an unmet need to restore blood flow to ischemic tissues while avoiding detrimental inflammation and other side effects in a minimally-invasive format for the 50% of patients with PAD that are ineligible for surgical interventions. While other studies have used VEGF, FGF, PDGF, GM-CSF, MCP-1, or bFGF in animal models of PAD, only bFGF has been used thus far in clinical trials. The first of these trials showed no adverse effects in the short term study; however, the second trial by Cooper et al., in 2001 found no positive effects with bFGF treatment and reported the negative side effect of severe proteinuria (excess proteins excreted in urine). For these reasons the study was terminated prematurely. The third clinical trial did report increased peak walking time without increased incidence of death or cardiac events in patients treated with bFGF, but also noted the high incidence of proteinuria.

Biomaterial systems have also been used to control the delivery of bFGF via gelatin microspheres in a phase 1 clinical trial. While this trial demonstrated promising results of improved perfusion and transcutaneous oxygen pressure, no placebo controls were used in this study to verify these findings. Other randomized clinical trials of bFGF administration in PAD patients have not demonstrated improvement vs. placebo controls. In addition, the synthesis of the gelatin microspheres required the use of glutaraldehyde e a highly cytotoxic crosslinking agent. The injectable microgel system used in our study avoids toxic agents and instead utilizes biocompatible polymer systems consisting of PEG and PCL which can be tuned for controlled peptide release without the need for chemical crosslinking Our study also reduces the cost of treatment by utilizing economical peptides in lieu of costly proteins such as bFGF. One explanation for the limited success of the use of single growth factors may be that PAD treatments are complicated by the interplay between angiogenesis and inflammation in this pathogenesis. Careful regulation of inflammation and angio-genesis is needed to treat PAD as some level of inflammation is needed for the initiation of angiogenesis to promote collateral vessel formation and restore blood flow to ischemic tissues. In this study we used small peptides proangiogenic C16 and anti-inflammatory Ac-SDKP e which take into consideration for controlling both angiogenic and inflammatory responses in PAD. The incorporation of this dual peptide treatment into an injectable polymer microgel proved to promote recovery of ischemic hind limbs in a mouse model of PAD while minimizing inflammatory responses.

The current study used LDPI and OCT imaging techniques to image blood flow and perfusion recovery in the model of PAD. These methods are advantageous over traditional imaging methods such as MRI and CT as they can be performed non-invasively in vivo and do not require a contrast agent. LDPI is non-invasive and semi-quantitative, with its ease of use making LDPI the gold standard for measuring recovery from hind limb ischemia. OCT is more sensitive than LDPI, with a higher resolution; however it is also depth limited. While LDPI could only accurately measure surface perfusion (within 200 mm) in the footpad, OCT can be used to image blood vessels up to 2 mm in depth of the ischemic calf muscle. According to results from LDPI, OCT, fluorescent microangiography, histological approaches, and Vybrant phagocy-tosis assays, the perfusion recovery and inflammatory activation with peptide-loaded implantable scaffolds were similar to trends seen with multiple 1 mL injections of peptide-loaded microgels. Specifically, C16 and C16 in combination with Ac-SDKP restored perfusion in the hind limb to the highest levels of all treatments tested. Ac-SDKP decreased perfusion compared to no peptide treatment. Phagocytic activity and macrophage infiltration also increased with C16 compared to no peptide treatment whereas Ac-SDKP decreased these inflammatory responses. The combination of C16 and Ac-SDKP maintained the low levels of phagocytic activity and macrophage infiltration observed with Ac-SDKP treatment alone.

Injectable polymer microgels provide an effective method for delivering functional peptides to the site of ischemia. Microgels were fabricated from biocompatible, biodegradable, combinatorial polymers PEG and PCL (FIG. 1A). The sole gel transition of the injectable polymer microgels used in this study allows for easy mixing of peptides into the polymer solution at room temperature. When injected to a target site, the peptides are stably encapsulated in a gel at the body temperature, keeping them in close proximity to the site of arterial blockage. The rapid sole gel transition observed with the 21% PEG-79% PCL polymer minimizes the flow of injectable microgel and a loss of peptides. As compared to peptides delivered in PBS solution, injectable polymer microgels resulted in greater effects on angiogenesis and inflammation, indicating the need of our injectable polymer microgels to retain peptides at the site of ischemia for improved therapeutic efficiency. However, peptide delivery via a single bulk injection of polymer microgel did not alter angiogenesis or inflammation as significantly as multiple, small volume injections of microgels (10, 1 mL injections). Small volume microgel injections may have released peptides more rapidly due to the increased surface area to volume ratio, as evident by IVIS imaging of peptide release (FIG. 1De E). The PBS injection may release peptides too quickly to have prolonged effects on angiogenesis or inflammation. Multiple small volume injections of polymer microgel provide an ideal time course for peptide release in this model.

Peptides were used for this study in lieu of growth factors due to their lower cost. Without loading in microgels, peptides injected in PBS did not significantly alter any of the measured outcomes, indicating the need for microgels to sustain release of peptides to the surrounding tissue. When incorporated into polymer microgels, anti-inflammatory Ac-SDKP decreased phagocytic activity and macrophage infiltration, successfully minimizing the host inflammatory response to the polymer microgels and avoiding potential aggravation of inflammatory activated endothelium in occluded blood vessels. However, Ac-SDKP treatment alone slightly decreased angiogenesis or perfusion in the hind limb, suggesting the treatment of Ac-SDKP alone is not suitable for restoring function to ischemic limbs affected by PAD. Pro-angiogenic C16 loaded microgels increased angiogenesis and perfusion to the ischemic hind limb; however they also resulted in increased inflammation with increased phagocytic activity and macrophage infiltration compared to no peptide treatment. This high level of inflammatory response is concerning when considering translating these therapies to human patients with inflamed arteries. Therefore, a pro-angiogenic treatment without inflammatory exacerbation was sought. Microgels loaded with C16b Ac-SDKP resulted in increased blood perfusion to the ischemic hind limb, as evaluated by LDPI, OCT, and fluorescent microangiography, as well as limited inflammatory response as evaluated by Vybrant phagocytosis, histology, and F4/80 staining. The treatment with pro-angiogenic C16 in combination with anti-inflammatory Ac-SDKP provided optimal collateral angiogenesis without detrimental inflammation, suggesting an ideal treatment for PAD by regulating angiogenesis and inflammation independently. Quantification of perfusion capacity and phagocytic activity directly correlated with results obtained from peptide-loaded scaffolds in a subcutaneous model, which confirms our previous work. The site-specific delivery of these peptides prevents unintended vascularization or inflammatory reduction in other tissues e such as retinal neovascularization or reduction of alveolar macrophage activity. At the site of ischemia, however, blood flow was increased, fibrosis and detrimental inflammation (as measured by phagocytic activity and macrophage infiltration) were minimized, and tissue necrosis was prevented by our minimally-invasive, site-specific delivery of the therapeutic peptides.

A mechanism of uncoupling angiogenesis and inflammation by co-treatment of C16 and Ac-SDKP was investigated in vitro. The overall expression level of MMP-9 in Raw 264.7 was higher than that of mAECs (FIG. 5A, D), and the overall expression level of TIMP-1 in Raw 264.7 was negligible compared to that of mAECs (FIG. 5B, E), indicating that macrophages might be a major regulator of MMP-9 activation in vivo. However, the numbers of F4/80 positive cells (FIG. 2Ge J) and angiogenic endothelial cells forming vessels (FIGS. 3 and 4) at the injection sites of the mouse PAD model varied significantly among the peptide conditions. Considering these facts, it is suggested that MMP-9 activation regulated by its gene expression and TIMP-1 together with the number of cells producing MMP-9 played a cooperative role in modulating angiogenic response at the ligation sites in vivo. When TNF-a inhibitors were co-treated with peptides, MMP-9 activation did not change in both macrophages and endothelial cells with intact tubulogenesis, but the phagocytic activity of macrophages significantly decreased. On the other hand, MMP-9 inhibition significantly reduced tubulo-genic activity of endothelial cells. Taken together, these results suggest that the inflammatory effects of peptide treatments were mediated by TNF-a secretion, while the angiogenic effects were mediated by MMP-9 activity. We also demonstrated that the regulation of inflammation through TNF-α was independent of MMP-9 mediated angiogenesis.

These findings are consistent with a recent study by Camargo et al. which proved independent modulation of TNF-a without affecting NF-kb, a transcription factor for MMP-9. Many other factors are known to regulate MMP-9 besides TNF-a, including the inflammatory cytokines IL-1b and IL-1a. In fact, in a pivotal study by Bond et al., IL-1b was proven to be a more potent promoter of MMP-9 than TNF-a. Without the synergistic effects of PDGF or bFGF, TNF-a did not significantly stimulate MMP-9 activity, indicating the need for combined cytokines and growth factors to stimulate maximal MMP-9 secretion. However, IL-1a alone did stimulate low levels of MMP-9 activity, and even higher levels when combined with PDGF or bFGF. TNF-a and IL-1a activate NF-kb, whereas bFGF and PDGF activate the ERK-1/ERK-2 MAPK pathway resulting in activation of AP-1, another transcription factor of MMP-9. As explained by Bond et al., their results indicate that both AP-1 and NF-kb are required for MMP-9 activation. The binding regions of these promoters are proximal to each other, allowing for interaction. Therefore multiple signal transduction pathways are needed for MMP-9 expression, with either TNF-a or IL-b required for NF-kb activation. In our study, inflammation was modulated independently of angiogenesis. MMP-9 expression was maintained during inhibition of TNF-a, possibly due to alternate mechanisms of NF-kb activation by IL-1 and/or AP-1 stimulation by PDGF or bFGF. The independent control of angiogenesis and inflammation should contribute to clinical translation of our approach as an optimal PAD treatment.

### Example 2

This embodiment of the present invention relates to a temperature-sensitive, self-adhesive hydrogel to deliver iPSC-derived cardiomyocytes for tissue repair. The example is further described in Wang et al., International Journal of Cardiology 190 (2015) 177-180, the contents of which are incorporated herein by reference.

Induced pluripotent stem cells (iPSCs) from patients' somatic tissues provide a viable source to create autologous cardiomyocytes (CMs) for potential cardiac-related cell therapies. However, a gap between the generation of iPSC-derived cardiomyocytes (iPSC-CMs) and the successful intra-cardiac engraftment of the cells to restore heart function remains to be bridged. Clinical data reporting engraftment of cells within human heart tissue has not been without its challenges, with significant cell loss from the site of delivery due to the physical stress of the cardiac cycle and the hostile inflammatory response within the infarct zone. Hydrogels have been proven to support the survival of multiple cell types and have served as a platform for cell transplantation. Yet, the use of tissue-adhesive, temperature-sensitive hydrogels to deliver iPSC-derived cardiomyocytes to infarcted heart remains to be explored. Therefore, we developed a polymer hydrogel to encapsulate, deliver, and integrate iPSC-CMs into infarcted myocardium to restore heart function.

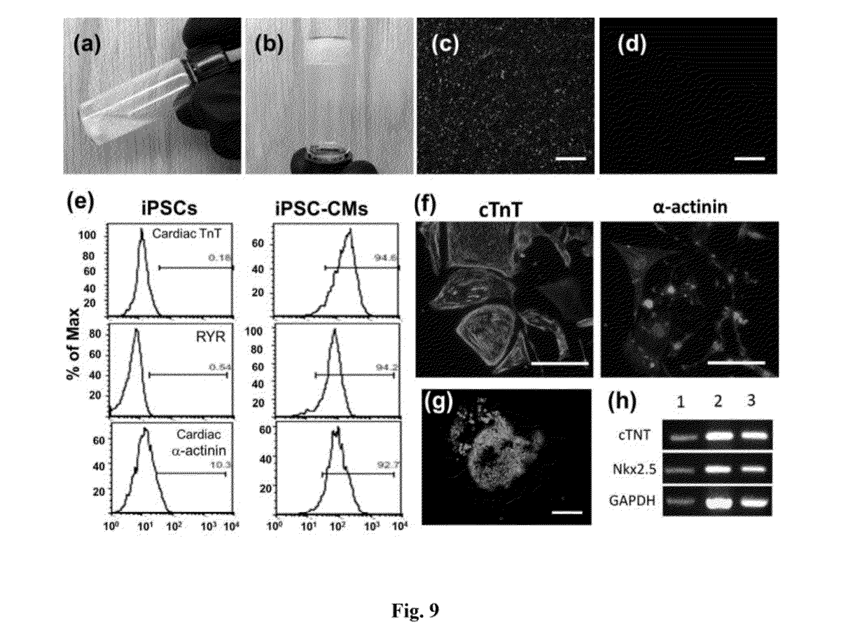

A temperature-sensitive biodegradable copolymer (polyethylene glycol-co-poly-8-caprolactone (PEG-PCL)) was synthesized and conjugated with a collagen-binding peptide (SYIRIADTNIT). The polymer was soluble in aqueous solutions at room temperature and underwent solution-to-gel transition at 37° C. (FIGS. 9 a,b). As the peptide-modified polymer had a strong affinity to collagen I in vitro (FIG. 9c), it was expected that it would significantly increase the binding of the hydrogel to an infarcted heart, thus immobilizing iPSC-CMs within the damaged myocardium. Functional, beating cardiomyocytes were derived from patient fibroblast-derived iPSCs using a “Matrigel sandwich” method. The cardiac differentiation was confirmed by fluorescence-activated cell sorting (FACS) and immunostaining with positive expression of cardiac markers cardiac troponin T (cTNT), ryanodine receptor 2 (RYR) and α-actinin (FIG. 9f). The iPSC-CMs encapsulated in the polymer hydrogel at 37° C. for two weeks maintained their viability (FIG. 9g) and cardiac phenotype, as evidenced by strong expression of troponin T and Nkx2.5 (FIG. 9h); thus, PEG-PCL does not appear to have obvious toxic effects on iPSC-CMs.

Engraftment of iPSC-CMs to infarcted myocardium using the peptide-modified hydrogel and potential improvement on infarcted heart function and structure were assessed with a rat myocardial infarction (MI) model. All animal surgery and animal care were approved by the Institutional Animal Care and Use Committee (IACUC) at Vanderbilt University (protocol: M/12/074). The left anterior descending (LAD) coronary artery of nude rats was ligated to induce MI. 30 minutes post-MI, iPSC-CMs alone, or modified copolymer solution with or without iPSC-CMs (2-4 million/rat) were injected around the infarct border zone. A negative control group received the LAD ligation and phosphate-buffered saline (PBS) injection without cells or copolymer.

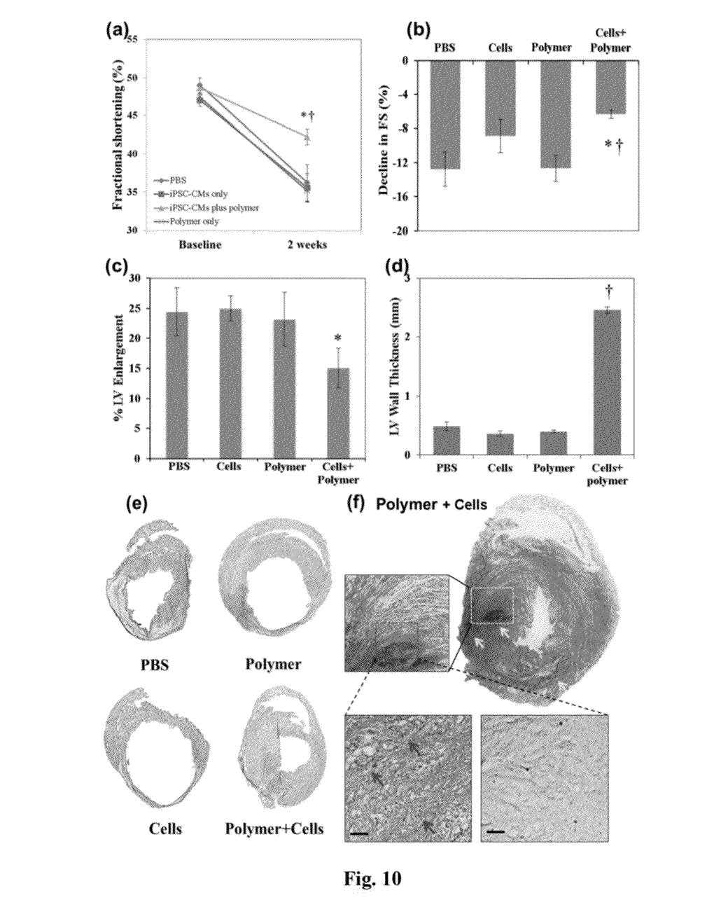

At two weeks post-injection, heart dimensions and functional output were assessed by echocardiography. All groups had ventricular dilation and reduced fractional shortening (FS) (FIG. 10). However, rats treated with iPSC-CM-encapsulated copolymer demonstrated significantly less decline in FS (Δ=−6.37±0.49%) compared to other groups (Δ=−12.77±2.04, −11.44±2.04 and −12.65±1.53 for PBS control, iPSC-CM only, and polymer only groups, respectively (FIG. 10a). p=0.016 vs. PBS, p=0.021 vs. iPSC-CM only, and p=0.005 vs. polymer only). Overall, the iPSC-CM plus polymer group demonstrated 50.1%, 28.2% and 49.6% improvement in LV systolic function over PBS, iPSC-CM only and polymer only groups, respectively (FIG. 2b). Moreover, rats treated with iPSC-CM plus polymer demonstrated a trend toward less LV enlargement (Δ=15.07±3.24%) compared to other groups (A=24.44±3.99%, 25.02±2.03% and 23.17±4.51% for PBS control, iPSC-CM only, and polymer only groups, respectively; FIG. 10c) although this reached statistical significance only in comparison to the iPSC-CM group (p=0.032). Overall, iPSC-CM plus polymer group demonstrated 38.3%, 39.8 and 35.0% less LV enlargement over PBS, iPSC-CM only and polymer only groups, respectively, suggesting that iPSC-CM encapsulated in polymer curtailed adverse ventricular remodeling better than other treatment modalities.

Histological examination of the hearts was performed and the LV wall thickness of each group was measured using ImageJ and averaged based on 3 randomly selected spots each rat. Result demonstrated that, in addition to LV chamber enlargement, the LAD ligation resulted in dramatic thinning and significant fibrosis of the LV anterior free wall at two weeks in control groups (FIG. 10e). In contrast, heart injected with polymer-encapsulated iPSC-CMs had smaller LV chamber, thicker LV free wall and less fibrosis (FIG. 2e). Overall, hearts in the iPSC-CM plus polymer group were significantly thicker than all other groups; the average LV anterior wall thickness of cell plus polymer group was 2.46±0.06 mm, compared with 0.48±0.07, 0.35±0.05 and 0.39±0.02 mm in PBS, cell only and polymer only groups, respectively (FIG. 2d, p<0.001). In summary, implantation of iPSC-CMs encapsulated in polymer hydrogel was much more effective at limiting adverse LV remodeling and preserving cardiac function after MI than other treatment modalities. Importantly, as implantation of iPSC-CMs or polymer alone did not elicit as favorable outcomes as the iPSC-CMs plus polymer group, we attribute the latter group's synergistic effects to enhanced survival of transplanted iPSC-CMs in vivo. Consistent with this notion, staining for human nuclei confirmed the presence of iPSC-CMs, delivered with the polymer hydrogel, in the peri-infarct region of the host rat myocardium at 2 weeks (FIG. 2f); moreover, the implanted cells maintained their cardiac phenotype, as demonstrated by positive staining of cardiac α-actinin (FIG. 10f). By contrast, no human nuclei were detected in hearts of control groups at 2 weeks.

This Example shows a temperature-sensitive, collagen-binding hydrogel based system to deliver human iPSC-derived cardiomyocytes to improve cardiac structure and function in infarcted rat heart. Moreover, our studies indicate that the beneficial effects of encapsulating iPSC-CMs in hydrogel are mediated through enhanced survival of transplanted iPSC-CMs in vivo. While future studies are needed to demonstrate long-term functional engraftment of transplanted cells, our study illustrates a promising biomaterial-based approach to overcome a commonly recognized obstacle to the potentially revolutionary cell-based approaches to repair failing hearts: survival of donor cells in the infarcted heart.

Throughout this document, including references are mentioned. All such references are incorporated herein by reference, including the references set forth in the following list:

### REFERENCES

PLA-PEG-PLA and Its Electroactive Tetraaniline Copolymer as Multi-interactive Injectable Hydrogels for Tissue Engineering. Cui H, et al. Biomacromolecules, 2013.
Minimally invasive injectable short nanofibers of poly(glycerol sebacate) for cardiac tissue engineering. Ravichandran R, et al. Nanotechnology. 2012; 23: 385102.
Human embryonic stem cell-derived microvascular grafts for cardiac tissue preservation after myocardial infarction. Kraehenbuehl T P, et al. Biomaterials. 2011; 32: 1102-9.
Kalamajski S. et al. J Biol Chem. 2007; 282:16062.

### REFERENCES

1. D. Lloyd-Jones, R. Adams, M. Carnethon, G. De Simone, T. B. Ferguson, K. Flegal, E. Ford, K. Furie, A. Go, K. Greenlund, N. Haase S. Hailpern, M. Ho, V. Howard, B. Kissela, S, Kittner, D. Lackland, L. Lisabeth, A. Marelli, M. McDermott, J. Meigs, D. Mozaffarian, G. Nichol, C. O'Donnell, V. Roger, W. Rosamond, R. Sacco, P. Sorlie, R. Stafford; J. Steinberger, T. Thom, S. Wasserthiel-Smoller, N. Wong, J. Wylie-Rosett, Y. Hong, Heart disease and stroke statistics—2009 update; a report from the Americas Heart Association Statistics Committee and Stroke Statistics Subcommittee. Circulrition. 119, e21-181 (2009).
2. W. H. Frishman, P. Anversa, Stem cell therapy for myocardial regeneration: the future is now. Heart Dis 4, 205 (2002).
3. E. J, Birks, P. D. Tansley, J. Hardy, R. S. George. C. T. Bowles, M. Burke, N. R. Banner, A. Khaghani, M. H. Yacoub, Left ventricular assist device and drug therapy for the reversal of heart failure. N Engl J Med 355, 1873-1884 (2006).
4. M. S. Slaughter, J. G. Rogers, C. A. Milano, S. D. Russell; J. V. Conte; D. Feldman, B. Sun, A. J. Tatooles, R. M. Delgado, 3rd, J. W. Long, T. C. Wozniak, W. Ghumman, D. J. Farrar, O. H. Frazier, Advanced heart failure treated with continuous-flow left ventricular assist device. N Engl J Med 361, 2241-2251 (2009).
5. M. Gheorghlade, W. S. Colucci, K. Swedberg, Beta-blockers in chronic heart failure. Circulation 107, 1570-1575 (2003).
6. K. Miki, H. Uenaka, A. Saito, S. Miyagawa, T. Sakaguchi, T. Higuchi, T. Shimizu, T. Okano, S. Yamanaka, Y. Sawa, Bioengineered myocardium derived from induced pluripotent stem cells improves cardiac function and attenuates cardiac remodeling following chronic myocardial infarction in rats. Stem Cells Transi Med 1, 430-437 (2012).
7. D. K. Singla, X. Long, C. Glass, R. D. Singla, B. Yan, Induced pluripotent stem (iPS) cells repair and regenerate infarcted myocardium. Mol Pharm 8, 1573-1581 (2011).
8. P. A. Lalit, D. J. Hei, A. N. Raval, T. J. Kamp, Induced pluripotent stem cells for post-myocardial infarction repair: remarkable opportunities and challenges. Circ Res 114, 1328-1345 (2014).
9. W. Schierling, L. A. Kunz-Schughart, F. Muders, G. A. Riegger, D. P. Griese, Fates of genetically engineered haematopoietic and mesenchymal stem cell grafts in normal and injured rat hearts. J Tissue Eng Regen Med 2, 354-364 (2008).
10. I. M. Barbash, P. Chouraqul, J. Baron, M. S. Feinberg, S. Etzion; A. Tessone, L. Miller, E. Guetta, D. Zipori, L. H. Kedes, R. A. Kloner, J. Leor, Systemic delivery of bone marrow-derived mesenchymal stem cells to the infarcted myocardium: feasibility, cell migration, and body distribution. Circulation 108, 863-868 (2003).
11. M. Hofmann; K. C. Wollert, G. P. Meyer; A. Menke, L. Arseniev, B. Hertenstein, A. Ganser, W. H. Knapp, H. Drexler, Monitoring of bone marrow cell homing into the infarcted human myocardium. Circulation 111, 2198-2202 (2005).
12. T. Freyman, G. Polin, H. Osman, J. Crary, M. Lu, L. Cheng, M. Palasis, R. L. Wilensky, A quantitative, randomized study evaluating three methods of mesenchymal stem cell delivery following myocardial infarction. Eur Heart J 27, 1114-1122 (2006).
13. V. F. Segers, R. T. Lee, Stem-cell therapy for cardiac disease. Nature 451, 937-942 (2008).
14. B. Doyle, B. J. Kemp, P Chareonthaltawee, C. Reed, J. Schmeckpeper, P. Sorajja, S. Russell, P. Araoz, S. J. Riederer, N. M, Caplice, Dynamic tracking during intracoronary injection of 18F-FDG-labeled progenitor cell therapy for acute myocardial infarction. J Nucl Med 48, 1708-1714 (2007).
15. N. Landa, L. Miller, M. S. Feinberg, R. Holbova, M. Shachar, I. Freeman, S. Cohen, J. Leor, Effect of injectable alginate implant on cardiac remodeling and function after recent and old infarcts in rat. Circulation 117, 1388-1396 (2008).
16. K. L. Fujimoto, Z. Ma, D. M. Nelson, R. Hashizume, J. Guan, K. Tobita, W. R. Wagner, Synthesis, characterization and therapeutic efficacy of a biodegradable, thermoresponsive hydrogel designed for application in chronic infarcted myocardium. Biomaterials 30, 4357-4368 (2009).
17. K. L. Christman, A. J. Vardanian, O. Fang, R. E, Sievers, H. H. Fok, R. J. Lee, Injectable fibrin scaffold improves cell transplant survival, reduces infarct expansion, and induces neovasculature formation in ischemic myocardium. J Am Coll Cardiol 44, 654-660 (2004).
18. I. Kutschka, I. Y. Chen, T. Kofidis, T. Arai, G. von Degenfeld, A. Y. Sheikh, S. L. Hendry, J. Pearl, G. Hoyt, R. Sista, P. C. Yang, H. M. Blau, S. S. Gambhir, R. C. Robbins, Collagen matrices enhance survival of transplanted cardiomyoblasts and contribute to functional improvement of ischemic rat hearts. Circulation 114, 1167-173 (2006).
19. E. J. Suurornen, J. P. Veinot, S. Wong, V. Kapila, J. Price, M. Griffith, T. G. Mesana, M. Ruel, Tissue-engineered injectable collagen-based matrices for improved cell delivery and vascularization of ischemic tissue using CD133+ progenitors expanded from the peripheral blood. Circulation 114, 1138-144 (2006).
20. M. S. Kim, H. Hyun, G. Khang H. B. Lee, Preparation of thermosensitive diblock copolymers consisting of MPEG and polyesters. Macromolecules 39, 3099-3102 (2006).
21. M. S. Kim, K. S. Seo, G. Khang, S. H, Cho, H. B. Lee, Preparation of poly(ethylene glycol)-block-poly(caprolactone) copolymers and their applications as thermo-sensitive materials. Journal of Biomedical Materials Research Part A 70A, 154-158 (2004).
22. J. L. Murphy, L. Vollenweider, F. M. Xu, B. P. Lee, Adhesive Performance of Biomimetic Adhesive-Coated Biologic Scaffolds, Biomacromolecules 11, 2976-2984 (2010).
23. S. Kalamajski, A. Aspberg, A. Oldberg, The decorin sequence SYIRIADTNIT binds collagen type I. Journal of Biological Chemistry 282. 16062-16067 (2007).
24. J. Zhang, M. Klos, G. F. Wilson, A. M. Herman, X. Lian, K. K. Raval, M. R. Barron. L. Hou, A. G. Soerens, J. Yu, S. P. Palecek G. E. Lyons, J. A. Thomson, T. J. Herron, J. Jalife, T. J. Kamp, Extracellular matrix promotes highly efficient cardiac differentiation of human pluripotent stem cells: the matrix sandwich method. Circ Res. 111, 1125-1136 (2012).
25. T. Wang, D. Q. Wu, X. J. Jiang, X. Z. Zhang, X. Y. Li, J. F. Zhang, Z. B Zheng, R. Zhuo, H. Jiang, C. Huang, Novel thermosensitive hydrogel injection inhibits post-infarct ventricle remodelling. Eur J Heart Fail 11, 14-19 (2005).
26. J. M. Singelyn, K. L. Christman; Injectable materials for the treatment of myocardial infarction and heart failure; the promise of decellularized matrices. J Cardiovasc Transl Res 3, 478-486 (2010).
27. K. L. Christman, H. H. Fok, R. E. Sievers, Q. Fang, R. J. Lee, Fibrin glue alone and skeletal myoblasts in a fibrin scaffold preserve cardiac function after myocardial infarction. Tissue Eng 10, 403-409 (2004).
28. M. A. Laflamme, C. E. Murry: Heart regeneration. Nature 473, 326-335 (2011).
29. B. Assmus, V. Schachinger, C. Teupe, M. Britten, R. Lehmann, N. Dobert, F. Grunwald, A. Aicher, C. Urbich, H. Martin, D. Hoeizer, S. Dimmeler, A. M. Zeiher, Transplantation of Progenitor Cells and Regeneration Enhancement in Acute Myocardial infarction (TOPCARE-AMI). Circulation 106, 3009-3017 (2002).
30. D, Orlic, J. Kajstura, S. Chimenti, I. Jakoniuk, S. M. Anderson, B. Li, J. Pickel, R. McKay, B. Nadal-Ginard, D. M. Bodine, A. Lerl, P. Anversa, Bone marrow cells regenerate infarcted myocardium. Nature 410, 701-705 (2001).
31, H. Reinecke, C. E. Murry, Electromechanical coupling between skeletal and cardiac muscle: Implications for infarct repair. Circulation 98, 757-758 (1998).
32. W. Z. Zhu, K. D. Hauch, C. Xu, M. A. Laflamme, Human embryonic stem cells and cardiac repair. Transplant Rev (Orlando) 23, 53-68 (2009).
33. M. Fujiwara, P. S. Van, T. G. Otsuji, G. Narazaki, H. Uosaki, H. Fukushima, K. Kuwahara, M. Harada, H. Matsuda, S. Matsuoka, K. Okita, K. Takahashi, M. Nakagawa, T. Ikeda, R. Sakata, C. L. Mummery, N. Nakatsuji, S. Yamanaka, K. Nakao, J. K. Yamashita, Induction and Enhancement of Cardiac Cell Differentiation from Mouse and Human Induced Pluripotent Stem Cells with Cyclosporin-A. Plos One 6, (2011).
34. X. J. Lian, C. Hsiao, G. Wilson, K. X. Zhu, L. B. Hazeltine, S. M. Azarin, K. K. Raval, J. H. Zhang, T. J. Kamp, S. P. Palecek, Robust cardiomyocyte differentiation from human pluripotent stem cells via temporal modulation of canonical Wnt signaling. Proceedings of the National Academy of Sciences of the United States of America 109, E1848-E1857 (2012).
35. L. C. Amado, A. P. Saliaris, K. H. Schuleri, M. St John, J. S. Xie, S. Cattaneo, D. J. Durand, T. Fitton, J. Q. Kuang, G. Stewart, S. Lehrke, W. W. Baumgartner, B. J. Martin, A. W. Heldman, J. M. Hare, Cardiac repair with intramyocardial injection of allogeneic mesenchymal stem cells after myocardial infarction. Proc Natl Acad Sci USA 102, 11474-11479 (2005).
36. M. F. Berry, A. J. Engler, Y. J. Woo, T. J. Pirolli, L. T. Bish, V. Jayasankar, K. J. Morine, T. J. Gardner D. E. Discher, H. L. Sweeney, Mesenchymal stem cell injection after myocardial infarction improves myocardial compliance. Am J Physiol Heart Circ Physiol 290, H2196-2203 (2006).
37. I. M. Barbash, P. Chouraqui, J. Baron, M. S. Feinberg, S. Etzion, A. Tessone, L. Miller, E. Guetta, D. Zipori, L. H. Kedes, R. A. Kloner, J. Leor, Systemic delivery of hone marrow-derived mesenchymal stem cells to the infarcted myocardium—Feasibility, cell migration, and body distribution. Circulation 108, 863-868 (2003).
38. T. Freyman, G. Polin, H. Osman, J. Crary, M. M. Lu, L. Cheng, M. Palasis, R. L. Wilensky, A quantitative, randomized study evaluating three methods of mesenchymal stem cell delivery following myocardial infarction. European Heart Journal 27, 1114-1122 (2000.
39. C. H. Chen, M. Y. Chang, S. S. Wang, P. C. Hsieh, injection of autologous bone marrow cells in hyaluronan hydrogel improves cardiac performance after infarction in pigs. Am J Physiol Heart Circ Physiol 306, H1078-1086 (2014).
40. R. D. Levit, N. Landazuri, E. A. Phelps, M. E. Brown, A. J. Garcia, M. E. Davis, G. Joseph, R. Long, S. A, Safley, J. D. Suever, A. N. Lyle, C. J. Weber, W. R. Taylor, Cellular encapsulation enhances cardiac repair. J Am Heart Assoc 2, e000367 (2013).
41. W. N. Lu, S. H. Lu, H. B. Wang, D. X. Li, C. M. Duan, Z. Q. Liu, T. Hao, W. J. He, B. Xu, Q. Fu, Y. C. Song, X. H. Xie, C. Y. Wang, Functional improvement of infarcted heart by co-injection of embryonic stem cells with temperature-responsive chitosan hydrogel. Tissue Eng Part A 15, 1437-1447 (2009).
42. E. Mathieu, G. Lamirault, C. Toquet, P. Lhommet, E. Rederstorff, S. Sourice, K. Biteau, P. Hulin, V. Forest, P. Weiss, J. Guicheux, P. Lemarchand, Intramyocardial delivery of mesenchymal stem cell-seeded hydrogel preserves cardiac function and attenuates ventricular remodeling after myocardial infarction. Plos One 7, e51991 (2012).
43. H. Wang, Z. Liu, D. Li, X. Guo, F. K. Kasper, C. Duan, J. Zhou, A. G. Mikos, C. Wang, Injectable biodegradable hydrogels for embryonic stem cell transplantation: improved cardiac remodelling and function of myocardial infarction. J Cell Mol Med 16, 1310-1320 (2012).
44. H. Hyun, Y. H. Kim, I. B. Song, J. W. Lee, M. S. Kim, G. Khang, K. Park, H. B. Lee, In vitro and in vivo release of albumin using a biodegradable MPEG-PCL diblock copolymer as an in situ gel-forming carrier. Biomacromolecules 8, 1093-1100 (2007).
45. J. Y. Lee, K. S. Kim, Y. M. Kang, E. S. Kim, S. J. Hwang, H. B. Lee, B. H. Min, J. H. Kim, M. S. Kim, In vivo efficacy of paclitaxel-loaded injectable in situ-forming gel against subcutaneous tumor growth. Int J Pharm 392, 51-56 (2010).
46. P. J. Neame, C. J. Kay, D. J. McQuillan, M. P. Beales, J. R. Hassell, Independent modulation of collagen fibrillogenesis by decorin and lumican. Cell Mol Life Sci 57, 859-863 (2000).
47, S. F. Nagueh, G. Shah, Y. Wu, Torre-Amione, N. M. King, S. Lahmers, C. C. Witt, K Becker, S. Labeit, H. L. Granzier, Altered titin expression, myocardial stiffness, and left ventricular function in patients with dilated cardiomyopathy. Circulation 110, 155-152 (2004).
48. J. H. Omens, Stress and strain as regulators of myocardial growth. Prog Biophys Mol Biol 69, 559-572 (1998).
49. S. M. Weis, J. L. Emery, K. D. Becker, D. J. McBride, Jr., J. H. Omens, A. D. McCulloch, Myocardial mechanics and collagen structure in the osteogenesis imperfecta murine (oim). Circ Res 87, 663-669 (2000).
50. H. J. Cho, N. Lee, J. Y. Lee, Y. J. Chol, M. II, A. Wecker, J. O. Jeong, C. Curry, G. Qin, Y. S. Yoon, Role of host tissues for sustained humoral effects after endothelial progenitor cell transplantation into the ischemic heart. J Exp Med 204, 3257-3269 (2007).
51. M. Zhang, D. Methot, V. Poppa, Y. Fujio, K. Walsh, C. E. Murry, Cardiomyocyte grafting for cardiac repair: graft cell death and anti-death strategies, J Mol Cell Cardiol 33, 907-921 (2001).
52. M. A. Laflamme, J. Gold, C. Xu, M. Hassanipour, E. Rosler, S. Police, V. Muskheli, C. E. Murry, Formation of human myocardium in the rat heart from human embryonic stem cells. Am J Pathol 167, 663-671 (2005).
53. M. A. Laflamme, K. Y. Chen, A. V. Naumova, V. Muskhell, J. A. Fugate, S. K. Dupras, H. Reinecke, C. Xu, M. Hassanipour, S. Police, C. O'Sullivan, L. Collins, Y. Chen, E. Minami, E. A. Gill, S. Ueno, C. Yuan, J. Gold, C. E. Murry, Cardiomyocytes derived from human embryonic stem cells in pro-survival factors enhance function of infarcted rat hearts. Nat Biotechnol 25, 1015-1024 (2007).

We claim:

1. A polymeric compound a co-polymer microgel at least one bioactive agent.

2. The compound of claim 1, wherein the microgel comprises monomethoxypoly(ethylene glycol)-co-poly(ε-caprolactone) (mPEG-PCL).

3. The compound of claim 1, wherein the mPEG is about 750 Da.

4. The compound of claim 1, wherein the microgel comprises about 21% PEG-b-79% PCL.

5. The compound of claim 1, wherein bioactive agent is at least one pro-angiogenic peptide and at least one anti-inflammatory peptide.

6. The compound of claim 5, wherein the pro-angiogenic peptide is C16 and the anti-inflammatory peptide is Ac-SDKP.

7. The compound of claim 1, wherein the microgel comprises a co-polymer of the following formula: 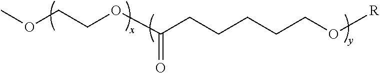
wherein x is 10-50% and y is 90-50%, and R is the bioactive agent.

8. The compound of claim 7, wherein the bioactive agent is at least one pro-angiogenic peptide and at least one anti-inflammatory peptide.

9. The compound of claim 7, wherein the bioactive agent selectively binds to fibrous tissue.

10. The compound of claim 7, wherein the bioactive agent includes a decorin-derived functional peptide.

11. The compound of claim 1, wherein the composition includes a transition temperature of about 20° C. to about 50° C.

12. The compound of claim 11, wherein the transition temperature is about mammalian body temperature.

13. The compound of claim 1, wherein the bioactive agent is at least one cell.

14. The compound of claim 13, wherein the at least one cell are stem cells.

15. A composition comprising a polymeric compound and a pharmaceutically acceptable carrier, wherein the polymeric compound comprises a polymer microgel at least one bioactive agent.

16. The composition of claim 15, wherein the polymeric compound is of the following formula: 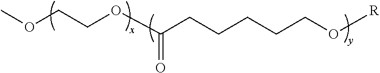
wherein x is 10-50% and y is 90-50%, and R is the bioactive agent.

17. The composition of claim 1, wherein the composition includes a transition temperature of about 20° C. to about 50° C.

18. The composition of claim 17, wherein the transition temperature is about body temperature.

19. The composition of claim 1, wherein the pharmaceutically acceptable carrier is in injection carrier.

20. A method for treating damaged tissue to a patient in need thereof, comprising:
providing a composition comprising a polymeric compound and a pharmaceutically acceptable carrier, wherein the polymeric compound is of the following formula: 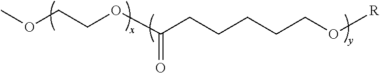
wherein x is 10-50% and y is 90-50%, and R is the bioactive agent;
administering by injection an effect amount of the composition.
